(19) United States
(12) Patent Application Publication
(10) Pub. No.: US 2012/0198576 A1
(43) Pub. Date: Aug. 2, 2012

(54) METHODS FOR MAKING EMBRYONIC CELLS, EMBRYOS, AND ANIMALS SENSITIZED TO STRESS

(75) Inventors: Lawrence J. Wangh, Auburndale, MA (US); Cristina Hartshorn, Needham, MA (US); Yanwei Jia, Waltham, MA (US)

(73) Assignee: BRANDEIS UNIVERSITY, Waltham, MA (US)

(21) Appl. No.: 13/378,436

(22) PCT Filed: Jul. 1, 2010
(86) PCT No.: PCT/US2010/040701

§ 371 Date: Apr. 17, 2012

Related U.S. Application Data
(60) Provisional application No. 61/222,588, filed on Jul. 2, 2009.

(51) Int. Cl. C12N 5/0735; A01K 67/027; A61K 31/343; A61B 17/435; A61K 33/30; A61K 31/198; A61K 31/4545; A61K 31/7072; C12Q 1/68; A61K 31/395

(52) U.S. Cl. 800/9; 435/375; 435/366; 435/6.12; 514/468; 514/183; 424/641; 514/564; 514/318; 514/50; 600/34

(57) ABSTRACT

Embodiments of the invention are based upon the discovery that exposure of cleavage-stage embryos to a stress inducer, e.g. heat shock or chemical, renders the exposed embryos more sensitive to a secondary treatment with a stress inducer, e.g. heat shock or chemical inducer. Accordingly, the present invention is directed to methods for making embryos, embryonic cells arising from them, and animals and plants that are sensitized to stress, e.g. physiologic or chemical stressors. Methods of screening for inducers and inhibitors of stress using, as test model systems, embryonic cells, embryos, animals, and plants that are sensitized to stress are also disclosed.

### CROSS-REFERENCE TO RELATED APPLICATIONS

This application claims the benefit under 35 U.S.C. §119(e) of U.S. Provisional Application No. 61/222,588, filed Jul. 2, 2009, the content of which is incorporated herein by reference in its entirety.

### GOVERNMENT SUPPORT

This invention was made with Government support with a sub-award No. P50 HG002360 awarded by the National Institutes of Health Subcontract from Center of Excellence in Genomic Science. The U.S. Government has certain rights to the invention.

### FIELD OF THE INVENTION

Embodiments of the present invention are directed to methods for making embryos, embryonic cells arising from them, and animals that are sensitized to stress, e.g. physiologic or chemical stressors. Methods of screening for inducers and inhibitors of stress using, as test model systems, embryonic cells, embryos, and animals that are sensitized to stress are also disclosed.

### BACKGROUND OF THE INVENTION

Over the past thirty years, more than 1% of births in the US and as many as 4% in Denmark¹ have been initiated using some form of in vitro fertilization (IVF). There is now increasing concern and investigation into whether such procedures have long-term consequences that may or may not be obvious at birth^{4,5}. Indeed, recent studies suggest that in-vitro fertilization (IVF) is associated with some rare genetic syndromes such as Beckwith-Wiedemann syndrome^{6,7} and Angelman Syndrome⁸. An increased risk of several birth defects in IVF babies was also reported^{9,10}.

IVF includes a wide variety of treatments and protocols and no rigorous investigations of altered gene expression in preimplantation human embryos are available for any of them. However, both short-term and long-term effects of environmental conditions on preimplantation mouse embryos have been analyzed. Ecker et al² transferred genetically marked blastocysts grown in vivo, along with blastocysts cultured under different conditions in vitro, into the same foster mother and then tested the resulting sibling mice for their capacities to perform in a maze. The results clearly show that mice born from embryos cultured in vitro were not as maze-bright as mice derived from embryos grown in vivo. Moreover, mice obtained from cultured embryos lacked the caution and fear that normal mice are born with. At the molecular level, genome wide patterns of DNA methylation and gene expression were different in embryos cultured in vitro for a few days as compared to those grown in vivo to the equivalent stages of development^{11,12,2}.

Accordingly, it would be important to discover the reasons for such phenomena as there exists a significant need to provide improved methods for growing embryos and embryonic stem cells to avoid the observed problems.

### SUMMARY OF THE INVENTION

We have discovered that exposure of cleavage-stage embryo cells to a stress inducer such as heat shock or a stressor (e.g., a chemical stressor), renders the exposed embryo cells more sensitive to a secondary, even milder, stress treatment (e.g., heat). The stress sensitivity, such as to heat, of the exposed cells persists through multiple rounds of cell division, e.g., when the embryo cells are transplanted, throughout embryonic development, and longer. This is in contrast to what has been observed previously in post-implantation mouse embryos, where a primary exposure of a post-implantation embryo to heat protects it from a second more severe heat shock, i.e. thermotolerance occurs.

In particular, we have found that cleavage-stage embryonic cells exposed to both primary and secondary heat shock contain approximately twice the amount of heat shock protein mRNA, i.e. hsp70i, after secondary treatment than the levels found in embryos only exposed to one treatment. The significant increase in mRNA levels upon secondary treatment occurred after hsp70i mRNA levels were allowed to return to normal subsequent to the primary treatment. In addition, the increase in hsp70i mRNA occurred whether or not the secondary treatment was given in the same or subsequent embryonic stage, e.g. 8-cell embryos or blastocysts. Thus, we have observed, at the molecular level, a heat shock “memory” that occurs after a primary stress, e.g. heat or chemical stress, that makes embryos more responsive, i.e. more sensitive, to a secondary chemical or heat stress. While not to be bound by theory, we believe that primary heat treatment triggers a change in DNA methylation with a concomitant change in chromatin structure within the promoters of these genes and that this epigenetic change persists in daughter cells, rendering them more sensitive to secondary heat treatment.

Accordingly, we now provide methods for making sensitized embryos or embryonic cells. The methods involve treating a cleavage staged embryo or embryonic cell arising from a cleavage stage embryo with a stress inducer such that the levels of a heat shock protein or mRNA are increased to a sufficient level to exhibit a greater response to a second treatment with a stress inducer. When the stress inducer is heat shock, the heat shock is applied in vitro or ex vivo. The stressor can also be a chemical compound, in which case, the stress inducer can be applied in vitro or in vivo, such as in utero.

In one embodiment, the methods for making sensitized embryos or embryonic cells further comprises subjecting the cleavage staged embryo or embryonic cell arising from said embryo to a second treatment with a stress inducer.

In another embodiment one tests the sensitized embryonic cells in vitro. For example in wells, such as plastic wells.

Heat shock proteins are known indicators of stress. The levels of any heat shock protein can be measured in methods of the invention. In one embodiment, the level of a heat shock protein selected from Hsp10, Hsp27, HspB1, Hsp40, Hsp60, Hsp71, Hsp70, Hsp72, Grp78 (BiP), Hsx70, HSP70i, Hsp90, Grp94, Hsp104, or Hsp110, is measured. In one embodiment, the level of heat shock proteins selected from GroES, GrpE, DnaJ, GroEL, 60 kDa antigen, DnsK, HtpG, C62.5, ClpB, ClpA, and ClpX, is measured.

The treatment with a stress inducer can be performed in vitro or in vivo (i.e. in utero).

In one embodiment, the stress inducer is heat shock. Heat shock can be achieved in vitro by raising the incubation temperature of a culture containing cleavage stage embryos/cells a few degrees above the species specific physiological optimal temperature for a limited period of time. As one versed in the art will appreciate, the precise amount of heat and length of time will be species dependent and can be determined experimentally by using several temperatures and several times. In the case of mammalian embryos to a temperature in the range of about 40° C. to about 45° C. In one embodiment the heat shock is achieved by raising the incubation temperature to 43° C. from 37° C. In one embodiment, heat shock is administered for 10-25 minutes. In one embodiment, heat shock is achieved in vitro by raising the temperature 4°-6° C. above the optimal incubation temperature used for normal development of the embryo or embryonic cell, optimal incubation temperatures are well known to those of skill in the art and can be found in for example, in “Molecular Methods in Developmental Biology; Xenopus and Zebrafish” By Matthew Guille, Edition: 2, v. 127, 1999, Humana Press, Totowa, N.J. 07512; and “Developmental Biology Protocols: Volume III” (Methods in Molecular Biology) by Rocky and Cecilia W. Lo, Humana Press, Totowa, N.J. 07512. In one embodiment, heat shock is achieved by raising the temperature to a temperature in the range of about 38° C. to about 45° C.

Any desired number of embryo's or cells can be treated. In one embodiment, 1-32 embryonic cells are treated.

In some embodiments, the embryo is a human embryo, in some embodiments the embryo is a non-human embryo. In some embodiments the embryo is a non-human mammal, such as a mouse, a rat, a horse, a cow, a pig, a sheep, a cat, a dog, a non-human primate, or any other non-human mammal. The embryo may also be from a plant.

In methods of the invention, the levels of heat shock protein can be measured by assessing the levels of heat shock protein or the level of heat shock protein mRNA. In one embodiment, the levels of heat shock protein are measured by assessing the levels of a reporter gene product (e.g. a visibly detectable protein) operably linked to a heat shock protein promoter.

In one embodiment, the stress inducer used in methods of the invention is a chemical stress inducer. Any chemical inducer of stress can be used. A variety of chemical stress inducers are known to those of skill in the art. In one embodiment, the chemical stress inducer is selected from bimoclomol (BIM), 2-Cyclopenten-1-one, thapsigargin; tunicamycin, a geldanamycin derivative, zinc, an amino acid analog, α-difluoromethylornithine, a household chemical, or an environmental pollutant.

Also provided are methods for making sensitized test animals. The methods involve a) treating a cleavage staged embryo with a stress inducer such that the levels of a heat shock protein or mRNA are increased to a sufficient level to exhibit a greater response to a second treatment with a stress inducer (when the stress inducer is heat shock, the heat shock is applied in vitro); b) developing the cleavage staged embryo of step (a) for implantation; and c) implanting the embryo of step (b) into the uterus of an animal for in-vivo growth and birth, whereby the birthed animal is exhibits a sensitized response to a secondary stress inducer.

In one embodiment, the method for making a sensitized test animal further comprises subjecting the cleavage staged embryo to a second treatment with a stress inducer, wherein the second treatment is performed either in vitro or in vivo.

Methods for screening for compounds that inhibit the induction of stress by a stress inducer are also provided. The methods involve using a test model selected from a sensitized test animal, or a sensitized embryo, or embryonic cell of the invention are also provided. Method steps include i) contacting a test model with a test compound ii) contacting the test model with a stress inducer, ii) assessing the level of heat shock protein or mRNA in cells of the test model, and iii) comparing the level of heat shock protein or mRNA determined to a control level. The control level is a level of heat shock protein or mRNA observed in cells of a corresponding test model treated with the same stress inducer as the test model, but not treated with the test compound. A decreased level of heat shock protein or mRNA as compared to the control level indicates that the test compound is an inhibitor of the stress inducer.

The invention also encompasses methods for screening for compounds that induce stress. The methods involve i) contacting a test model selected from a sensitized test animal, or a sensitized embryo, or embryonic cell of the invention with a test compound, then contacting the test model with a stress inducer, ii) assessing the level of heat shock protein or mRNA in cells of the test model; and iv) comparing the level of heat shock protein or mRNA determined to a control level. The control level is a level of heat shock protein or mRNA observed in cells of a corresponding test model treated with the same stress inducer as the test model, but not treated with the test compound. An increased level of heat shock protein or mRNA in the test model as compared to the control level indicates that the test compound is an inducer of stress.

### BRIEF DESCRIPTION OF THE DRAWINGS

FIGS. 1a-1b. FIG. 1a shows a graph of hsp70i mRNA transcription in single cells isolated from 8-cell stage embryos after 20 minutes of heat shock and different recovery times; the bars show average hsp70i mRNA+DNA levels per cell plus standard deviation; X-axis: recover time (hours), Y Axis: DNA+RNA/cell. FIG. 1b shows a graph of the average hsp70i mRNA transcription in single cells from 8-cell stage embryos after heat shock of different duration and two hours of recovery; X-axis: Heat shock duration (minutes), Y Axis: DNA+RNA/cell.

FIG. 2 shows a graph of the average number of cells per embryo, 24 hours after heat shock at the 8-cell stage for different lengths of time; X-axis: Heat shock duration (minutes), Y Axis: number of cells/embryo.

FIGS. 3a-3b. FIG. 3a shows a graph depicting double heat shock effect on hsp70i expression in single cells from embryos at the 8-cell stage. X-axis: Non-preheated (light grey? Solid outline) and preheated (dark grey? Dotted outline), Y Axis: DNA+RNA/cell. The light grey bar shows average hsp70i mRNA+DNA levels in embryos that had been heat-shocked only once, for 10 minutes at the 8-cell stage (cultured 24 hours). The dark grey bar shows average hsp70i mRNA+DNA levels in embryos that had been preheated for 20 minutes at the 8-cell stage (cultured 24 hours) and then heated again for 10 minutes after a 4-hour recovery period. FIG. 3b shows a graph depicting long term double heat shock effect in blastocyst stage mouse embryos. X-axis: Non-preheated (light grey? Solid outline) and preheated (dark grey? Dotted outline), Y Axis: DNA+RNA/cell. The light grey bar shows average hsp70i mRNA+DNA levels in embryos heat-shocked for 10 minutes at the blastocyst stage. The dark grey bar shows average hsp70i mRNA+DNA levels in embryos preheated for 20 minutes at 8-cell stage, then heat-shocked again for 10 minutes at the blastocyst stage.

FIGS. 4A and 4B show a Real-time LATE-PCR standard curve for hsp70i quantification, obtained using mouse genomic DNA at known copy numbers. FIG. 4A) shows the PCR amplification curves, X-axis cycle number Y-axis: fluorescence units for 4000 copies, 400 copies, 40 copies, 4 copies and No-Template Control (NTC) respectively from left to right. FIG. 4B) shows a graph depicting the correlation between CT values (Y-axis) and copy numbers (X-axis).

FIG. 5 shows a graph depicting hsp70i DNA+mRNA copy numbers in single cells isolated from 8-cell stage embryos heat-shocked for 30 minutes at 43° C. or non-heat-shocked embryos. Y-axis: DNA+RNA/cell, X-axis: HS, heat shock; NHS, no heat shock; RT, RT-PCR; NRT, no reverse transcriptase in the PCR mix. The average copy numbers are shown above the columns (black lines=standard deviation).

FIG. 6 shows the levels of hsp70i mRNA two hours after heat shock at 43° C. recovered from five equivalent samples containing ˜500 cells. The levels of hsp70i mRNA correlate with the length of heat shock treatment. The longer the heat shock, the higher the hsp70i mRNA levels. In the absence of heat shock there were an average of 105±6 copies hsp70i mRNA. Twenty minutes of heat shock increased the level of hsp70i mRNA 353±14 copies, while 30, 40 and 50 minutes heat shock raised the levels of hsp70i mRNA to 601±34, 691±9 and 875±103 copies, respectively.

FIG. 7 shows that an average of 246±41 copies of hsp70i mRNA were measured in the absence of heat shock (Group 1), while a 20-minute heat shock resulted in 555±91 copies (Group 2). Seven days after the first 20 minutes heat shock, the level of hsp70i mRNA was, 94±7 copies (Group 4), which means that there was no mRNA left over from the first heat shock. However, a second 20-minute heat shock seven days after the first heat shock generated 1,070±134 copies (Group 3), almost double the number of hsp70i mRNA molecules produced by a single heat shock. This increased number of mRNA molecules is not left over mRNA due to the first heat shock, or is it dues to a greater number of cells in the sample. Instead, it indicates that some of the cells remembered the experience of the first heat shock and had a higher response to the second heat shock.

### DETAILED DESCRIPTION OF THE INVENTION

We have discovered that exposure of embryos to a stress inducer, e.g. heat shock or chemical, renders the exposed embryos more sensitive to a secondary treatment with a stress inducer, e.g. heat shock or chemical inducer. We have determined this by observing that levels of heat shock protein mRNA are significantly increased in “cleavage-stage” embryos exposed to both primary and secondary heat shock, even after mRNA levels were allowed to return to normal after the primary treatment. The increase in heat shock protein mRNA occurred whether or not the secondary treatment was given in the same or subsequent embryonic stage, e.g. 8-cell embryos or blastocysts. Thus, we have discovered, at the molecular level, a heat shock “memory” that occurs after application of an external primary stress, e.g. heat or chemical stress, that makes embryos more responsive, i.e. more sensitive, to a secondary chemical or heat stress delivered after the primary response has returned to background levels and the cells that were stressed have divided.

While not to be bound by theory, we believe that this primary heat treatment triggers a change in DNA methylation with a concomitant change in chromatin structure within the promoters of these genes and that this epigenetic change persists in daughter cells, rendering them more sensitive to secondary heat treatment.

Embodiments of the present invention are based on the newly discovered heat shock “memory” in cleavage-stage embryos. Methods for making embryos, embryonic cells arising from them, and animals that are sensitized to stress, e.g. physiologic or chemical stressors are provided. Embodiments of the invention further encompass methods of using the embryonic cells, embryos and animals that are sensitized to stress as test model systems to screen for both inducers and inhibitors of stress.

For convenience, certain terms used in the entire application (including the specification, examples, and appended claims) are defined specifically as follows. Unless defined otherwise, all technical and scientific terms used herein have the same meaning as commonly understood by one of ordinary skill in the art to which this invention belongs.

It should be understood that this invention is not limited to the particular methodology, protocols, and reagents, etc., described herein and as such may vary. The terminology used herein is for the purpose of describing particular embodiments only, and is not intended to limit the scope of the present invention, which is defined solely by the claims.

Other than in the operating examples, or where otherwise indicated, all numbers expressing quantities of ingredients or reaction conditions used herein should be understood as modified in all instances by the term “about.” The term “about” when used to described the present invention, in connection with percentages means ±1%, when used to describe degrees Celsius is ±1 degree.

In one respect, the present invention relates to the herein described compositions, methods, and respective component(s) thereof, as essential to the invention, yet open to the inclusion of unspecified elements, essential or not (“comprising”). In some embodiments, other elements to be included in the description of the composition, method or respective component thereof are limited to those that do not materially affect the basic and novel characteristic(s) of the invention (“consisting essentially of”). This applies equally to steps within a described method as well as compositions and components therein. In other embodiments, the inventions, compositions, methods, and respective components thereof, described herein are intended to be exclusive of any element not deemed an essential element to the component, composition or method (“consisting of”).

All patents, patent applications, and publications identified are expressly incorporated herein by reference for the purpose of describing and disclosing, for example, the methodologies described in such publications that might be used in connection with the present invention. These publications are provided solely for their disclosure prior to the filing date of the present application. Nothing in this regard should be construed as an admission that the inventors are not entitled to antedate such disclosure by virtue of prior invention or for any other reason. All statements as to the date or representation as to the contents of these documents is based on the information available to the applicants and does not constitute any admission as to the correctness of the dates or contents of these documents.

As used herein the term “sensitized” refers to the phenomenon of an increased responsiveness to a stressor (stress inducer) as measured by a statistically significant increase in expression level (e.g. statistically significant increase in measured protein or mRNA levels) of a heat shock protein in response to the stressor as compared to normal pre-sensitization levels, i.e. the expression level of the heat shock protein present after only one exposure to a stress inducer.

As used herein the term “stress inducer” or “stressor” refers to any compound or any physiological stress (e.g. heat, shear stress, cold, oxygen level, glucose level, etc.) that causes a cell to express one or more heat shock proteins. For example, heat shock protein expression is triggered by exposure of animals or cells to different kinds of environmental stress conditions, such as infection, inflammation, exercise, exposure to chemical toxins (e.g. ethanol, arsenic, trace metals and ultraviolet light, household chemicals), starvation, hypoxia (oxygen deprivation), heat, and water deprivation. Consequently, the heat shock proteins are also referred to as “stress proteins.”

A common stress inducer is heat shock, for example exposure of mammalian cells to temperatures ranging from about 40° C. to about 45° C., usually in a time frame of about 10 to about 25 minutes.

Examples of chemical stress inducers include, but are not limited to, bimoclomol (BIM); 2-cyclopenten-1-one; benzodiazepinones such as thapsigargin and tunicamycin; geldanamycin derivatives such as 17-DMAG and 17-AAG; zinc; amino acid analogs such as ethyl-a-nitro-/3-(3-indole)-propionate, (3-(2-indolyl)-DL-alanine, 4-methyl-DL-tryptophan, 6-methy 1-DL-tryptophan, 5-fluoro-DL-tryptophan, allylglycine, methallylglycine, methoxinine, ethionine, cysteic acid, o-methyl-aspartic acid, a-methyl-asparagine, 3-(2-thienyl)-DL-alanine, 3-(3-thienyl)-DL-alanine, 3-(2-furyl)-DL-alanine, 3-(p-fluorophenyl)-DL-alanine, (o-fluorophenyl)-DL-alanine, 0-(nz-fluorophenyl)-DL-alanine, 3-(p-chlorophenyl)-DL-alanine, 3-(o-chlorophenyl)-DL-alanine, 3-(m-chlorophenyl)-DL-alanine, 0-(p-bromophenyl)-DL-alanine, 3-(o-bromophenyl)-DL-alanine, 3-(m-bromophenyl)-DL-alanine, 3-(m-iodophenyl)-DL-alanine, 3-(p-iodophenyl)-DL-alanine, 3-(1-naphthyl)-DL-alanine, □-methyl-DL-phenylalanine, a-(phenyl)-DL-alanine, 7-(phenyl)-DL-o-amino-n-butyric acid, 0-(3,5-dihydroxyphenyl)-DL-alanine, N-acetyl-DL-phenylalanine, N-chloroacetyl-DL-phenylalanine, N-dichloroacetyl-DL-phenylalanine, N-carbethoxy-DL-phenylalanine, N-methyl-DL-phenylalanine, N-hydroxyethyl-DL-phenylalanine, N-(o-iodobenzoyl)-DL-phenylalanine, N-cyanoethyl-(3,4-dihydr, α-difluoromethylornithine, household chemicals such as bleach; and environmental pollutants, e.g. benzene.

One can use the chemicals in established or experimental concentrations that typically range from nanomolar concentrations to millimolar concentrations. A skilled artisan can readily determine the amount of any stressor based on well known art and additionally based on routine experimentation. For example, the stress inducing chemicals can be used from concentrations ranging from 10 nm to 1000 mM, 10 nM to 100 mM, 10-100 nM, 10-500 nM, 50-100 nM, 100-500 nM, 1-100 □M, 10-100 □M, 50-100 □M, 50-500 □M, 500-1000 □M, 1-100 mM, 10-100 mM, 50-100 mM, 50-500 mM, or 100-1000 mM. For example, sodium salicylate as a stress inducing agent, can be used between about 5-120 mM, 5-30 mM, 5-60 mM, 30-60 mM, 30-120, and 60-120 mM concentrations, for example at about 5, about 30, about 60 or about 120 mM concentrations. Cadmium chloride can be used in e.g., 50-200 □M, 50-100 □M, or 100-200 □M concentrations, for example at 50, 100 or 200 □M concentrations. Sodium arsenate can be used, for example at about 10-500 □M, such as at about 100 □M, about 200 □M, or about 300 □M concentrations. Isopropyl b-D-thiogalactopyranoside can be used, e.g., from 100 □M to 100 mM concentrations.

Additional known cell stressors include ambient particular matter, such as organic diesel exhaust particle chemicals, ethanol, glucose starvation followed by feeding, and anoxia followed by oxygenation. Conditions for exposing cells to these stressors are known and can be further tested using routine experimentation.

As used herein the term “secondary stress inducer” refers to a stress inducer used subsequently to a first treatment with a stress inducer, for example in a “secondary treatment.” Secondary stress induction can occur any time after a primary induction. Preferably secondary stress induction occurs after the embryo's or cells have recovered from the primary treatment. In one embodiment the secondary treatment is milder than the first treatment, e.g. a lower dosage of a chemical stress inducer or a lower temperature, or duration, of heat shock is used as compared to the primary treatment. In one embodiment the time for recovery from the primary treatment is determined by i) measuring the expression level of a heat shock protein or its mRNA prior to exposure to a stressor (primary treatment), ii) measuring the expression level of the heat shock protein or its mRNA after exposure to the primary treatment, and iii) at multiple time points after the primary treatment measuring the expression level of the heat shock protein or its mRNA. Embryo's or cells have recovered from a primary treatment when the measured levels of heat shock protein return to the levels present before primary treatment.

It should be noted that the first stress inducer and the second or secondary stress inducer can be the same but do not have to be the same, or even of the same class of stress inducers. One can for example use heat as a first inducer and heat as the second inducer. However, one can also have heat or anoxia or glucose deprivation as the first stress inducer and the second stress inducer can be a chemical or vice versa. Alternatively, the first stress inducer can be a first chemical and the second stress inducer can be a second, different chemical.

As used herein a “cleavage-staged embryo” refers to an embryo within the stages after fertilization of a one-cell embryo where the embryo undergoes a series of cell divisions, ultimately leading to formation of a hollow sphere of cells known as a blastocyst or blastula.

Cells from virtually all organisms respond to a variety of stresses by the rapid synthesis of a highly conserved set of polypeptides termed heat shock proteins (HSPs). There is considerable evidence that these stress proteins are essential for survival at both normal and elevated temperatures. HSPs also appear to play a critical role in the development of thermotolerance and protection from cellular damage associated with stresses such as ischemia, cytokines, and energy depletion. As used herein the term “heat shock protein” (HSP) is intended to encompass all known heat shock proteins and those that have yet to be discovered. Heat shock proteins are present in mammalian and non-mammalian animals. Non-limiting examples of mammalian heat shock proteins include, but are not limited to, Hsp10, Hsp27, HspB1, Hsp40, Hsp60, Hsp71, Hsp70, Hsp72, Grp78 (BiP), Hsx70, HSP70i, Hsp90, Grp94, Hsp104, and Hsp110. Non-limiting examples of non-mammalian heat shock proteins include, GroES, GrpE, DnaJ, GroEL, 60 kDa antigen, DnsK, HtpG, C62.5, ClpB, ClpA, and ClpX. Table 1 sets forth additional HSPs.

| | | | |
|---|---|---|---|
| | | | Example |
| | | | antibodies |
| | | | and |
| | | | proteins |
| | | | available on |
| | | | the date of |
| | | | filing from, |
| | | | e.g., Assay |
| Name | Synonyms | Entrez Gene ID; SEQ ID NO | Designs ™ |

Hsp70 Family

| | | | |
|---|---|---|---|
| APG-1 | HspA4L, Osp94 | 22824; | |
| | | mRNA sequence (SEQ ID NO: 4): | |
| | | NM_014278.2 | |
| | | PRT sequence (SEQ ID NO: 5): | |
| | | NP_055093.2 | |
| APG-2 | HspA4, Irp94, | 3308 | |
| | Hsp70RY | mRNA sequence (SEQ ID NO: 6): | |
| | | NM_002154.3 | |
| | | PRT sequence (SEQ ID NO: 7): | |
| | | NP_002145.3 | |
| BIP/Grp78 | HspA5, Hsp70-5 | 3309 | Antibodies |
| | | mRNA sequence (SEQ ID NO: 8): | (SPA-826, |
| | | NM_005347.3 | SPA-827), |
| | | PRT sequence (SEQ ID NO: 9): | Protein |
| | | NP_005338.1 | (SPP-765) |
| Grp75/ | MtHsp70, HspA9, | 3313 | Antibody |
| Mortalin | Hsp70-9, MOT, Mot-2 | mRNA sequence (SEQ ID NO: 10): | (SPS-825) |
| | | NM_004134.6 | |
| | | PRT sequence (SEQ ID NO: 11): | |
| | | NP_004125.3 | |
| Hsc70 | HspA8, Hsp70-8, | 3312 | Antibodies |
| | Hsp73, Hsc71 | mRNA sequences (SEQ ID NOS 12-13): | (SPA-815, |
| | | NM_006597.3 | SPA-816), |
| | | NM_153201.1 | Proteins |
| | | PRT sequences (SEQ ID NOS 14-15): | (SPP-751, |
| | | NP_006588.1 | SPP-752) |
| | | NP_694881.1 | |
| Hsp70-4 | Hsp70L1 HspA14 | 51182 | |
| | | mRNA sequence (SEQ ID NO: 16): | |
| | | NM_016299.2 | |
| | | PRT sequence (SEQ ID NO: 17): | |
| | | NP_057383.2 | |
| Hsp70B | HspA7 | 3311 | |
| | | mRNA sequence (SEQ ID NO: 18): | |
| | | NR_024151.1 | |
| | | PRT sequence (SEQ ID NO: 19): | |
| | | P48741 | |
| Hsp70B′ | HspA6, Hsp70-6 | 3310 | ELISA |
| | | mRNA sequence (SEQ ID NO: 20): | (EKS-725), |
| | | NM_002155.3 | Antibodies |
| | | PRT sequence (SEQ ID NO: 21): | (SPA-756, |
| | | NP_002146.2 | SPA-754), |
| | | | Protein |
| | | | (SPP-762) |
| Hsp70-Hom | HspA1L, Hum70t, | 3305 | |
| | Hsp70-1t | mRNA sequence (SEQ ID NO: 22): | |
| | | NM_005527.3 | |
| | | PRT sequence (SEQ ID NO: 23): | |
| | | NP_005518.3 | |
| Hsp72 | HspA1A, Hsp70-1a, | 3303 | ELISA |
| | Hsp70-1 | mRNA sequence (SEQ ID NO: 24): | (EKS-700), |
| | | NM_005345.5 | Antibodies |
| | | PRT sequence (SEQ ID NO: 25): | (SPA-810, |
| | | NP_005336.3 | SPA-812), |
| | | | Proteins |
| | | | (ESP-555, |
| | | | SPP-758) |
| Hsp72 | HspA1B, Hsp70-1b, | 3304 | ELISA |
| | Hsp70-1 | mRNA sequence (SEQ ID NO: 26): | (EKS-700), |
| | | NM_005346.4 | Antibodies |
| | | PRT sequence (SEQ ID NO: 27): | (SPA-810, |
| | | NP_005337.2 | SPA-812), |
| | | | Proteins |
| | | | (ESP-555, |
| | | | SPP-758) |
| Hsp110 | Hsp105, HspH1 | 10808 | Antibodies |
| | | mRNA sequence (SEQ ID NO: 28): | (SPA-1101, |
| | | NM_006644.2 | SPA-1103) |
| | | PRT sequence (SEQ ID NO: 29): | |
| | | NP_006635.2 | |
| HspA2 | Hsp70-2 | 3306 | Protein |
| | | mRNA sequence (SEQ ID NO: 30): | (ESP-502) |
| | | NM_021979.3 | |
| | | PRT sequence (SEQ ID NO: 31): | |
| | | NP_068814.2 | |
| HYOU1 | Hypoxia upregulated | 10525 | |
| | 1, Grp170, ORP 150 | mRNA sequences (SEQ ID NOS 32-33): | |
| | | NM_001130991.1 | |
| | | NM_006389.3 | |
| | | PRT sequences (SEQ ID NO: 34-35): | |
| | | NP_001124463.1 | |
| | | NP_006380.1 | |

Hsp70/Hsp40 Associated Co-chaperones & Others

| | | | |
|---|---|---|---|
| Bag-1 | HAP | 573 | Antibodies |
| | | mRNA sequences (SEQ ID NOS 36-37): | (AAM-400, |
| | | NM_001172415.1 | 905-735) |
| | | NM_004323.5 | |
| | | PRT sequences (SEQ ID NOS 38-39): | |
| | | NP_001165886.1 NP_004314.5 | |
| Bag-2 | NA | 9532 | |
| | | mRNA sequence (SEQ ID NO: 40): | |
| | | NM_004282.3 | |
| | | PRT sequence (SEQ ID NO: 41): | |
| | | NP_004273.1 | |
| Bag-3 | BIS | 9531 | |
| | | mRNA sequence (SEQ ID NO: 42): | |
| | | NM_004281.3 | |
| | | PRT sequence (SEQ ID NO: 43): | |
| | | NP_004272.2 | |
| Bag-4 | SODD | 9530 | |
| | | mRNA sequence (SEQ ID NO: 44): | |
| | | NM_004874.2 | |
| | | PRT sequence (SEQ ID NO: 45): | |
| | | NP_004865.1 | |
| Bag-5 | NA | 9529 | |
| | | mRNA sequences (SEQ ID NOS 46-48): | |
| | | NM_001015048.1 | |
| | | NM_001015049.1 | |
| | | NM_004873.2 | |
| | | PRT sequences (SEQ ID NOS 49-51): | |
| | | NP_001015048.1 NP_001015049.1 | |
| | | NP_004864.1 | |
| BAP | SIL1 | 64374 | |
| | | mRNA sequences (SEQ ID NOS 52-53): | |
| | | NM_001037633.1 | |
| | | NM_022464.4 | |
| | | PRT sequences (SEQ ID NOS 54-55): | |
| | | NP_001032722.1 NP_071909.1 | |
| CHIP | STUB1, UBOX1, | 10273 | |
| | HSPABP2 | mRNA sequence (SEQ ID NO: 56): | |
| | | NM_005861.2 | |
| | | PRT sequence (SEQ ID NO: 57): | |
| | | NP_005852.2 | |
| Hip | HspBP1 | 23640 | Antibody |
| | | mRNA sequences (SEQ ID NOS 58-59): | (SPA-766), |
| | | NM_001130106.1 | Protein |
| | | NM_012267.4 | (SPP-767) |
| | | PRT sequences (SEQ ID NOS 60-61): | |
| | | NP_001123578.1 NP_036399.3 | |
| Hop | STIP1, STI1 | 10963 | Antibody |
| | | mRNA sequence (SEQ ID NO: 62): | (SRA-1500), |
| | | NM_006819.2 | Protein |
| | | PRT sequence (SEQ ID NO: 63): | (SRP-1510) |
| | | NP_006810.1 | |

Hsp40/DnaJ Family

| | | | |
|---|---|---|---|
| CSP | Cysteine String | 80331 | Antibody |
| | Protein, DnaJC5 | mRNA sequence (SEQ ID NO: 64): | (VAP- |
| | | NM_025219.1 | SV003) |
| | | PRT sequence (SEQ ID NO: 65): | |
| | | NP_079495.1 | |
| CSP-beta | DnaJC5B | 85479 | |
| | | mRNA sequence (SEQ ID NO: 66): | |
| | | NM_025219.1 | |
| | | PRT sequence (SEQ ID NO: 67): | |
| | | NP_079495.1 | |
| CSP-gamma | DnaJC5G | 285126 | |
| | | mRNA sequence (SEQ ID NO: 68): | |
| | | NM_173650.1 | |
| | | PRT sequence (SEQ ID NO: 69): | |
| | | NP_775921.1 | |
| DnaJA4 | NA | 55466 | |
| | | mRNA sequences (SEQ ID NOS 70-72): | |
| | | NM_001130182.1 NM_001130183.1 | |
| | | NM_018602.3 | |
| | | PRT sequences (SEQ ID NOS 73-75): | |
| | | NP_001123654.1 NP_001123655.1 | |
| | | NP_061072.3 | |
| DnaJA5 | NA | 134218 | |
| | | mRNA sequences (SEQ ID NOS 76-77): | |
| | | NM_001012339.2 NM_194283.3 | |
| | | PRT sequences (SEQ ID NO: 78-79): | |
| | | NP_001012339.2 NP_919259.3 | |
| DnaJB12 | NA | 54788 | |
| | | mRNA sequences (SEQ ID NO: 80-81): | |
| | | NM_001002762.2 | |
| | | NM_017626.4 | |
| | | PRT sequences (SEQ ID NO: 82-83): | |
| | | NP_001002762.2 | |
| | | NP_060096.3 | |
| DnaJB13 | TSARG6 | 374407 | |
| | | mRNA sequence (SEQ ID NO: 84): | |
| | | NM_153614.2 | |
| | | PRT sequence (SEQ ID NO: 85): | |
| | | NP_705842.2 | |
| DnaJB14 | NA | 79982 | |
| | | mRNA sequence (SEQ ID NO: 86): | |
| | | NM_001031723.2 | |
| | | PRT sequence (SEQ ID NO: 87): | |
| | | NP_001026893.1 | |
| DnaJB8 | NA | 165721 | |
| | | mRNA sequence (SEQ ID NO: 88): | |
| | | NM_153330.2 | |
| | | PRT sequence (SEQ ID NO: 89): | |
| | | NP_699161.1 | |
| DnaJC11 | FLJ10737 | 55735 | |
| | | mRNA sequence (SEQ ID NO: 90): | |
| | | NM_018198.3 | |
| | | PRT sequence (SEQ ID NO: 91): | |
| | | NP_060668.2 | |
| DnaJC16 | NA | 23341 | |
| | | mRNA sequence (SEQ ID NO: 92): | |
| | | NM_015291.2 | |
| | | PRT sequence (SEQ ID NO: 93): | |
| | | NP_056106.1 | |
| DnaJC17 | NA | 55192 | |
| | | mRNA sequence (SEQ ID NO: 94): | |
| | | NM_018163.2 | |
| | | PRT sequence (SEQ ID NO: 95): | |
| | | NP_060633.1 | |
| DnaJC18 | NA | 202052 | |
| | | mRNA sequence (SEQ ID NO: 96): | |
| | | NM_152686.2 | |
| | | PRT sequence (SEQ ID NO: 97): | |
| | | NP_689899.1 | |
| DnaJC19 | TIM14 | 131118 | |
| | | mRNA sequences (SEQ ID NOS 98-99): | |
| | | NM_001190233.1 | |
| | | NM_145261.3 | |
| | | PRT sequences (SEQ ID NOS 100-101): | |
| | | NP_001177162.1 | |
| | | NP_660304.1 | |
| DnaJC4 | HSPF2; MCG18 | 3338 | |
| | | mRNA sequence (SEQ ID NO: 102): | |
| | | NM_005528.3 | |
| | | PRT sequence (SEQ ID NO: 103): | |
| | | NP_005519.2 | |
| DnaJC6 | DJC6 | 9829 | |
| | | mRNA sequence (SEQ ID NO: 104): | |
| | | NM_014787.2 | |
| | | PRT sequence (SEQ ID NO: 105): | |
| | | NP_055602.1 | |
| DnaJC7 | TPR2; TTC2 | 7266 | |
| | | mRNA sequences (SEQ ID NOS 106-107): | |
| | | NM_001144766.2 | |
| | | NM_003315.3 | |
| | | PRT sequences (SEQ ID NOS 108-109): | |
| | | NP_001138238.1 | |
| | | NP_003306.3 | |
| DnaJC8 | SPF31 | 22826 | |
| | | mRNA sequence (SEQ ID NO: 110): | |
| | | NM_014280.2 | |
| | | PRT sequence (SEQ ID NO: 111): | |
| | | NP_055095.2 | |
| DRIP78 | HDJ3; DnaJC14 | 85406 | |
| | | mRNA sequence (SEQ ID NO: 112): | |
| | | NM_032364.5 | |
| | | PRT sequence (SEQ ID NO: 113): | |
| | | NP_115740.5 | |
| ERdj3 | DnaJB11, ERj3, | 51726 | |
| | HEDJ; ABBP-2 | mRNA sequence (SEQ ID NO: 114): | |
| | | NM_016306.4 | |
| | | PRT sequence (SEQ ID NO: 115): | |
| | | NP_057390.1 | |
| ERdj5 | JPDI; DnaJC10 | 54431 | |
| | | mRNA sequence (SEQ ID NO: 116): | |
| | | NM_018981.1 | |
| | | PRT sequence (SEQ ID NO: 117): | |
| | | NP_061854.1 | |
| Hdj2 | DnaJA1, DJ2; DJA1; | 3301 | |
| | HSDJ; HSJ2 | mRNA sequence (SEQ ID NO: 118): | |
| | | NM_001539.2 | |
| | | PRT sequence (SEQ ID NO: 119): | |
| | | NP_001530.1 | |
| HdjC9 | JDD1; DnaJC9 | 23234 | |
| | | mRNA sequence (SEQ ID NO: 120): | |
| | | NM_015190.3 | |
| | | PRT sequence (SEQ ID NO: 121): | |
| | | NP_056005.1 | |
| Hlj1 | DnaJB4, DjB4; | 11080 | |
| | DnaJW | mRNA sequence (SEQ ID NO: 122): | |
| | | NM_007034.3 | |
| | | PRT sequence (SEQ ID NO: 123): | |
| | | NP_008965.2 | |
| HSC3 | DnaJB7 | 150353 | |
| | | mRNA sequence (SEQ ID NO: 124): | |
| | | NM_145174.1 | |
| | | PRT sequence (SEQ ID NO: 125): | |
| | | NP_660157.1 | |
| Hsc40 | DnaJB5 | 25822 | |
| | | mRNA sequences (SEQ ID NOS 126-128): | |
| | | NM_001135004.1 | |
| | | NM_001135005.1 | |
| | | NM_012266.4 | |
| | | PRT sequences (SEQ ID NOS 129-131): | |
| | | NP_001128476.1 | |
| | | NP_001128477.1 NP_036398.3 | |
| HscB | DnaJC20, Jac1, Hsc20 | 150274 | |
| | | mRNA sequence (SEQ ID NO: 132): | |
| | | NM_172002.3 | |
| | | PRT sequence (SEQ ID NO: 133): | |
| | | NP_741999.3 | |
| Hsj1 | DnaJB2, HSPF3 | 3300 | |
| | | mRNA sequences (SEQ ID NOS 134-135): | |
| | | NM_001039550.1 | |
| | | NM_006736.5 | |
| | | PRT sequences (SEQ ID NOS 136-137): | |
| | | NP_001034639.1 NP_006727.2 | |
| Hsp40/Hdj1 | DnaJB1, HSPF1 | 3337 | Antibodies |
| | | mRNA sequence (SEQ ID NO: 138): | (SPA-400, |
| | | NM_006145.1 | SPA-450), |
| | | PRT sequence (SEQ ID NO: 139): | Protein |
| | | NP_006136.1 | (SPP-400) |
| Htj1/ERdj1 | DNAJC1, DNAJL1, | 64215 | |
| | MTJ1 | mRNA sequence (SEQ ID NO: 140): | |
| | | NM_022365.3 | |
| | | PRT sequence (SEQ ID NO: 141): | |
| | | NP_071760.2 | |
| JDP1 | DnaJC12 | 56521 | |
| | | mRNA sequences (SEQ ID NOS 142-143): | |
| | | NM_021800.2 NM_201262.1 | |
| | | PRT sequences (SEQ ID NOS 144-145): | |
| | | NP_068572.1 NP_957714.1 | |
| MCJ | HSD18, DNAJD1, | 29103 | |
| | DNAJC15 | mRNA sequence (SEQ ID NO: 146): | |
| | | NM_013238.2 | |
| | | PRT sequence (SEQ ID NO: 147): | |
| | | NP_037370.2 | |
| Mdg1/ERdj4 | DnaJB9 | 4189 | |
| | | mRNA sequence (SEQ ID NO: 148): | |
| | | NM_012328.1 | |
| | | PRT sequence (SEQ ID NO: 149): | |
| | | NP_036460.1 | |
| MPP11 | MIDA1, DnaJC2, | 22791 | |
| | Zrf1, Zrf2 | mRNA sequence (SEQ ID NO: 150): | |
| | | NM_009584.4 | |
| | | PRT sequence (SEQ ID NO: 151): | |
| | | NP_033610.1 | |
| Mrj | DnaJB6, Hsj2; Msj1 | 10049 | |
| | | mRNA sequences (SEQ ID NOS 152-153): | |
| | | NM_005494.2 | |
| | | NM_058246.3 | |
| | | PRT sequences (SEQ ID NOS 154-155): | |
| | | NP_005485.1 | |
| | | NP_490647.1 | |
| P58(IPK) | DnaJC3, PRKRI; | 5611 | |
| | | mRNA sequence (SEQ ID NO: 156): | |
| | | NM_006260.3 | |
| | | PRT sequence (SEQ ID NO: 157): | |
| | | NP_006251.1 | |
| Rdj2 | DnaJA2, DJA2 | 10294 | |
| | | mRNA sequence (SEQ ID NO: 158): | |
| | | NM_005880.3 | |
| | | PRT sequence (SEQ ID NO: 159): | |
| | | NP_005871.1 | |
| RME-8 | DnaJC13 | 23317 | |
| | | mRNA sequence (SEQ ID NO: 160): | |
| | | NM_015268.3 | |
| | | PRT sequence (SEQ ID NO: 161): | |
| | | NP_056083.3 | |
| Tid1 | hTid-1, DnaJA3 | 9093 | |
| | | mRNA sequences (SEQ ID NOS 162-163): | |
| | | NM_001135110.1 | |
| | | NM_005147.4 | |
| | | PRT sequences (SEQ ID NOS 164-165): | |
| | | NP_001128582.1 | |
| | | NP_005138.3 | |

Hsp60/Hsp10 Family

| | | | |
|---|---|---|---|
| CCT2 | chaperonin containing | 10576 | |
| | TCP1 subunit 2 (beta), | mRNA sequence (SEQ ID NO: 166): | |
| | TCP1-beta, CCTB, | NM_006431.2 | |
| | CCT-beta; | PRT sequence (SEQ ID NO: 167): | |
| | | NP_006422.1 | |
| CCT3 | chaperonin containing | 7203 | |
| | TCP1 subunit 3 | mRNA sequences (SEQ ID NOS 168-170): | |
| | (gamma), CCTG, | NM_001008800.1 | |
| | CCT-gamma, TRIC5, | NM_001008883.1 | |
| | TCP1-gamma | NM_005998.3 | |
| | | PRT sequences (SEQ ID NOS 171-173): | |
| | | NP_001008800.1 NP_001008883.1 | |
| | | NP_005989.3 | |
| CCT4 | chaperonin containing | 10575 | |
| | TCP1 subunit 4 (delta), | mRNA sequence (SEQ ID NO: 174): | |
| | SRB, CCTd | NM_006430.2 | |
| | | PRT sequence (SEQ ID NO: 175): | |
| | | NP_006421.2 | |
| CCT5 | chaperonin containing | 22948 | |
| | TCP1 subunit 5 | mRNA sequence (SEQ ID NO: 176): | |
| | (epsilon), CCTE, CCT- | NM_012073.3 | |
| | epsilon, TCP1-epsilon | PRT sequence (SEQ ID NO: 177): | |
| | | NP_036205.1 | |
| CCT6A | chaperonin containing | 908 | |
| | TCP1 subunit 6A (zeta1), | mRNA sequences (SEQ ID NOS 178-179): | |
| | CCT6, CCTz, | NM_001009186.1 | |
| | HTR3, TCPZ, TCP20, | NM_001762.3 | |
| | MoDP-2, TTCP20, | PRT sequences (SEQ ID NOS 180-181): | |
| | CCT-zeta, CCT-zeta1, | NP_001009186.1 | |
| | TCP1-zeta | NP_001753.1 | |
| CCT6B | chaperonin containing | 10693 | |
| | TCP1 subunit 6B (zeta2), | mRNA sequence (SEQ ID NO: 182): | |
| | CCT-zeta2; TCP1- | NM_006584.2 | |
| | zeta2 | PRT sequence (SEQ ID NO: 183): | |
| | | NP_006575.2 | |
| CCT7 | chaperonin containing | 10574 | |
| | TCP1 subunit 7 (eta), | mRNA sequences (SEQ ID NOS 184-187): | |
| | HIV-1 Nef interacting | NM_001009570.2 | |
| | protein (Nip7-1), | NM_001166284.1 NM_001166285.1 | |
| | TCP1-eta, Ccth, CCT- | NM_006429.3 | |
| | Eta | PRT sequences (SEQ ID NOS 188-191): | |
| | | NP_001009570.1 NP_001159756.1 | |
| | | NP_001159757.1 NP_006420.1 | |
| Hsp10 | CPN10, GroES, | 3336 | Antibody |
| | HSPE1 | mRNA sequence (SEQ ID NO: 192): | (SPA-110), |
| | | NM_002157.2 | Protein |
| | | PRT sequence (SEQ ID NO: 193): | (SPP-110) |
| | | NP_002148.1 | |
| Hsp60 | HspD1, CPN60, | 3329 | ELISA |
| | GroEL, HSP65, | mRNA sequences (SEQ ID NOS 194-195): | (EKS-600), |
| | SPG13, HuCHA60 | NM_002156.4 NM_199440.1 | Antibodies |
| | | PRT sequences (SEQ ID NOS 196-197): | (SPA-806, |
| | | NP_002147.2 NP_955472.1 | SPA-829, |
| | | | SPA-828), |
| | | | Proteins |
| | | | (ESP-540, |
| | | | ESP-741, |
| | | | SPP-742) |
| TCP1 | CCT1, CCT-alpha, | 6950 | |
| | TCP1-alpha | mRNA sequences (SEQ ID NOS 198-199): | |
| | | NM_001008897.1 | |
| | | NM_030752.2 | |
| | | PRT sequences (SEQ ID NOS 200-201): | |
| | | NP_001008897.1 | |
| | | NP_110379.2 | |

Hsp90 Family

| | | | |
|---|---|---|---|
| Grp94 | Hsp90B1, Gp96 | 7184 | Antibodies |
| | | mRNA sequence (SEQ ID NO: 202): | (SPA-850, |
| | | NM_003299.1 | SPA-851, |
| | | PRT sequence (SEQ ID NO: 203): | SPA-827), |
| | | NP_003290.1 | Protein |
| | | | (SPP-766) |
| Hsp90 alpha | Hsp90AA1, Hsp86, | 3320 | ELISA |
| | Hsp89 | mRNA sequences (SEQ ID NOS 204-205): | (EKS-895), |
| | | NM_001017963.2 | Antibodies |
| | | NM_005348.3 | (SPS-771, |
| | | PRT sequences (SEQ ID NOS 206-207): | SPA-840), |
| | | NP_001017963.2 NP_005339.3 | Protein |
| | | | (SPP-766) |
| Hsp90 alpha | Hsp90AA2 | 3324 | |
| | | mRNA sequence (SEQ ID NO: 208): | |
| | | NG_005856 | |
| | | PRT sequence (SEQ ID NO: 209): | |
| | | Q14568 | |
| Hsp90 beta | Hsp90AB1 | 3326 | Antibodies |
| | | mRNA sequence (SEQ ID NO: 210): | (SPA-843, |
| | | NM_007355.2 | SPA-842) |
| | | PRT sequence (SEQ ID NO: 211): | |
| | | NP_031381.2 | |
| TRAP1 | Hsp75 | 10131 | |
| | | mRNA sequence (SEQ ID NO: 212): | |
| | | NM_016292.2 | |
| | | PRT sequence (SEQ ID NO: 213): | |
| | | NP_057376.2 | |

Hsp90 Associated Co-chaperones & Others

| | | | |
|---|---|---|---|
| Aha1 | Activator of Hsp90 | 10598 | |
| | ATPase homolog 1 | mRNA sequence (SEQ ID NO: 214): | |
| | | NM_012111.2 | |
| | | PRT sequence (SEQ ID NO: 215): | |
| | | NP_036243.1 | |
| Cdc37 | NA | 11140 | |
| | | mRNA sequence (SEQ ID NO: 216): | |
| | | NM_007065.3 | |
| | | PRT sequence (SEQ ID NO: 217): | |
| | | NP_008996.1 | |
| Hop | STIP1, STI1 | 10963 | Antibody |
| | | mRNA sequence (SEQ ID NO: 218): | (SRA-1500), |
| | | NM_006819.2 | Protein |
| | | PRT sequence (SEQ ID NO: 219): | (SRP-1510) |
| | | NP_006810.1 | |
| p23 | TEBP, PGE Synthase 3 | 10728 | Antibody |
| | | mRNA sequence (SEQ ID NO: 220): | (SPA-670), |
| | | NM_006601.5 | Protein |
| | | PRT sequence (SEQ ID NO: 221): | (SPP-670) |
| | | NP_006592.3 | |

Small Hsp Family (sHsps)

| | | | |
|---|---|---|---|
| Crystallin, | CRYAA, CRYA1, | 1409 | Antibody |
| alpha A | HSPB4 | mRNA sequence (SEQ ID NO: 222): | (SPA-221), |
| | | NM_000394.2 | Protein |
| | | PRT sequence (SEQ ID NO: 223): | (SPP-226) |
| | | NP_000385.1 | |
| Crystallin, | CRYAB, CRYA2, | 1410 | Antibodies |
| alpha B | HSPB5 | mRNA sequence (SEQ ID NO: 224): | (SPA-222, |
| | | NM_001885.1a | SPA-223, |
| | | PRT sequence (SEQ ID NO: 225): | SPA-225, |
| | | NP_001876.1 | SPA-226, |
| | | | SPA-227), |
| | | | Protein |
| | | | (SPP-227) |
| Heat shock | H11, E2IG1, HSP22, | 26353 | |
| 22 kDa protein 8 | HSPB8 | mRNA sequence (SEQ ID NO: 226): | |
| | | NM_014365.2 | |
| | | PRT sequence (SEQ ID NO: 227) | |
| | | NP_055180.1 | |
| heat shock | Hsp27, Hsp25, HSPB1 | 3315 | ELISA |
| 27 kDa protein 1 | | mRNA sequence (SEQ ID NO: 228): | (EKS-500), |
| | | NM_001540.3 | Antibodies |
| | | PRT sequence (SEQ ID NO: 229): | (SPA-801, |
| | | NP_001531.1 | SPA-803, |
| | | | SPA-800, |
| | | | SPA-525, |
| | | | SPA-523, |
| | | | SPA-524, |
| | | | 905-642), |
| | | | Proteins |
| | | | (NSP-510, |
| | | | ESP-715, |
| | | | SPP-716) |
| heat shock | HSPB2, MKBP | 3316 | |
| 27 kDa protein 2 | | mRNA sequence (SEQ ID NO: 230): | |
| | | NM_001541.3 | |
| | | PRT sequence (SEQ ID NO: 231): | |
| | | NP_001532.1 | |
| heat shock | HSPB3, HSPL27 | 8988 | |
| 27 kDa protein 3 | | mRNA sequence (SEQ ID NO: 232): | |
| | | NM_006308.1 | |
| | | PRT sequence (SEQ ID NO: 233): | |
| | | NP_006299.1 | |
| heat shock | cvHSP, HspB7 | 27129 | |
| 27 kDa protein | | mRNA sequence (SEQ ID NO: 234): | |
| family, member 7 | | NM_014424.4 | |
| (cardiovascular) | | PRT sequence (SEQ ID NO: 235): | |
| | | NP_055239.1 | |
| heat shock | HSPB6, Hsp20 | 126393 | Antibody |
| protein, alpha- | | mRNA sequence (SEQ ID NO: 236): | (SPA-769) |
| crystallin- | | NM_144617.1 | |
| related, B6 | | PRT sequence (SEQ ID NO: 237): | |
| | | NP_653218.1 | |
| heat shock | HSPB9, CT51 | 94086 | |
| protein, alpha- | | mRNA sequence (SEQ ID NO: 238): | |
| crystallin- | | NM_033194.2 | |
| related, B9 | | PRT sequence (SEQ ID NO: 239): | |
| | | NP_149971.1 | |
| outer dense | HSPB10, ODFP, | 4956 | |
| fiber of sperm | SODF, ODF1 | mRNA sequence (SEQ ID NO: 240): | |
| tails 1 | | NM_024410.3 | |
| | | PRT sequence (SEQ ID NO: 241): | |
| | | NP_077721.2 | |
| Crystallin, beta | CRYBA1 | 1411 | Antibody |
| A1 | | mRNA sequence (SEQ ID NO: 242): | (SPA-230; |
| | | NM_005208.4 | not known |
| | | PRT sequence (SEQ ID NO: 243): | which beta |
| | | NP_005199.2 | family |
| | | | members are |
| | | | recognized |
| | | | by this |
| | | | antibody), |
| | | | Proteins |
| | | | (SPP-235, |
| | | | SPP-236; |
| | | | not known |
| | | | which beta |
| | | | family |
| | | | members are |
| | | | represented |
| | | | in these |
| | | | native |
| | | | preparations) |
| Crystallin, beta | CRYBA2 | 1412 | |
| A2 | | mRNA sequence (SEQ ID NOS 244-246): | |
| | | NM_005209.1 | |
| | | NM_057093.1 | |
| | | NM_057094.1 | |
| | | PRT sequences (SEQ ID NOS 247-249): | |
| | | NP_005200.1 NP_476434.1 | |
| | | NP_476435.1 | |
| Crystallin, beta | CRYBA4 | 1413 | |
| A4 | | mRNA sequence (SEQ ID NO: 250): | |
| | | NM_001886.2 | |
| | | PRT sequence (SEQ ID NO: 251): | |
| | | NP_001877.1 | |
| Crystallin, beta | CRYBB1 | 1414 | |
| B1 | | mRNA sequence (SEQ ID NO: 252): | |
| | | NM_001887.3 | |
| | | PRT sequence (SEQ ID NO: 253): | |
| | | NP_001878.1 | |
| Crystallin, beta | CRYBB2 | 1415 | |
| B2 | | mRNA sequence (SEQ ID NO: 254): | |
| | | NM_000496.2 | |
| | | PRT sequence (SEQ ID NO: 255): | |
| | | NP_000487.1 | |
| Crystallin, beta | CRYBB3 | 1417 | |
| B3 | | mRNA sequence (SEQ ID NO: 256): | |
| | | NM_004076.3 | |
| | | PRT sequence (SEQ ID NO: 257): | |
| | | NP_004067.1 | |
| Crystallin, | CRYGA, CRYG1, | 1418 | Protein |
| gamma A | CRYG5 | mRNA sequence (SEQ ID NO: 258): | (SPP-240; |
| | | NM_014617.3 | not known |
| | | PRT sequence (SEQ ID NO: 259): | which |
| | | NP_055432.2 | gamma |
| | | | family |
| | | | members are |
| | | | represented |
| | | | in this native |
| | | | preparation) |
| Crystallin, | CRYGB, CRYG2 | 1419 | |
| gamma B | | mRNA sequence (SEQ ID NO: 260): | |
| | | NM_005210.3 | |
| | | PRT sequence (SEQ ID NO: 261): | |
| | | NP_005201.2 | |
| Crystallin, | CRYGC, CRYG3, | 1420 | |
| gamma C | CCL | mRNA sequence (SEQ ID NO: 262): | |
| | | NM_020989.3 | |
| | | PRT sequence (SEQ ID NO: 263): | |
| | | NP_066269.1 | |
| Crystallin, | CRYGD, CRYG4, | 1421 | |
| gamma D | CCP, PCC, CACA, | mRNA sequence (SEQ ID NO: 264): | |
| | CCA3 | NM_006891.3 | |
| | | PRT sequence (SEQ ID NO: 265): | |
| | | NP_008822.2 | |
| Crystallin, | CRYGN | 155051 | |
| gamma N | | mRNA sequence (SEQ ID NO: 266): | |
| | | NM_144727.1 | |
| | | PRT sequence (SEQ ID NO: 267): | |
| | | NP_653328.1 | |
| Crystallin | CRYGS, CRYG8 | 1427 | |
| gamma S | | mRNA sequence (SEQ ID NO: 268): | |
| | | NM_017541.2 | |
| | | PRT sequence (SEQ ID NO: 269): | |
| | | NP_060011.1 | |

Other Hsps and Chaperones

| | | | |
|---|---|---|---|
| Calnexin | CNX, P90 | 821 | Antibodies |
| | | mRNA sequences (SEQ ID NOS 270-271): | (SPA-860, |
| | | NM_001024649.1 | SPA-865) |
| | | NM_001746.3 | |
| | | PRT sequences (SEQ ID NOS 272-273): | |
| | | NP_001019820.1 NP_001737.1 | |
| Calreticulin | CRTC, ERp60, grp60 | 811 | Antibodies |
| | | mRNA sequence (SEQ ID NO: 274): | (SPA-600, |
| | | NM_004343.3 | SPA-601) |
| | | PRT sequence (SEQ ID NO: 275): | |
| | | NP_004334.1 | |
| CHIP | STUB1, UBOX1, | 10273 | |
| | HspABP2 | mRNA sequence (SEQ ID NO: 276): | |
| | | NM_005861.2 | |
| | | PRT sequence (SEQ ID NO: 277): | |
| | | NP_005852.2 | |
| HO-1 | heme oxygenase | 3162 | ELISA |
| | (decyclizing) 1, | mRNA sequence (SEQ ID NO: 278): | (EKS-800, |
| | HMOX1, Hsp32 | NM_002133.2 | EKS-810), |
| | | PRT sequence (SEQ ID NO: 279): | Antibodies |
| | | NP_002124.1 | (SPA-895, |
| | | | SPA-896, |
| | | | OSA-150, |
| | | | OSA-110, |
| | | | OSA-111), |
| | | | Proteins |
| | | | (SPP-730, |
| | | | SPP-732) |
| HO-2 | heme oxygenase | 3163 | Antibodies |
| | (decyclizing) 2, | mRNA sequences (SEQ ID NOS 280-283): | (OSA-200, |
| | HMOX2 | NM_001127204.1 | SPA-897), |
| | | NM_001127205.1 | Proteins |
| | | NM_001127206.1 | (NSP-550) |
| | | NM_002134.3 | |
| | | PRT sequences (SEQ ID NOS 284-287): | |
| | | NP_001120676.1 NP_001120677.1 | |
| | | NP_001120678.1 NP_002125.3 | |
| HSF-1 | HSTF1 | 3297 | Antibodies |
| | | mRNA sequence (SEQ ID NO: 288): | (SPA-950, |
| | | NM_005526.2 | SPA-901), |
| | | PRT sequence (SEQ ID NO: 289): | Protein |
| | | NP_005517.1 | (SPP-900) |
| HSF-2 | HSTF2 | 3298 | Antibody |
| | | mRNA sequences (SEQ ID NOS 290-291): | (SPA-960) |
| | | NM_001135564.1 NM_004506.3 | |
| | | PRT sequences (SEQ ID NOS 292-293): | |
| | | NP_001129036.1 NP_004497.1 | |
| Hsp47 | SerpinH1, Colligin, | 871 | Antibody |
| | Gp46 | mRNA sequence (SEQ ID NO: 294): | (SPA-470), |
| | | NM_001235.2 | Protein |
| | | PRT sequence (SEQ ID NO: 295): | (NSP-535) |
| | | NP_001226.2 | |
| KDELR1 | ERD2 | 10945 | Antibody |
| | | mRNA sequence (SEQ ID NO: 296): | (VAA- |
| | | NM_006801.2 | PT048) |
| | | PRT sequence (SEQ ID NO: 297): | |
| | | NP_006792.1 | |
| KDELR2 | ERD2.2 | 11014 | |
| | | mRNA sequences (SEQ ID NOS 298-299): | |
| | | NM_001100603.1 NM_006854.3 | |
| | | PRT sequences (SEQ ID NOS 300-301): | |
| | | NP_001094073.1 | |
| | | NP_006845.1 | |
| KDELR3 | ERD2L3 | 11015 | |
| | | mRNA sequences (SEQ ID NOS 302-303): | |
| | | NM_006855.2 | |
| | | NM_016657.1 | |
| | | PRT sequences (SEQ ID NOS 304-305): | |
| | | NP_006846.1 NP_057839.1 | |
| UGGT | UDP- | 56886 | Antibody |
| | glucose:glycoprotein | mRNA sequence (SEQ ID NO: 306): | (VAP- |
| | glucosyltransferase, | NM_020120.3 | PT068) |
| | HUGT1, UGCGL1, | PRT sequence (SEQ ID NO: 307): | |
| | GT, UGT1, UGTR | NP_064505.1 | |
| ERdj5 | JPDI; DnaJC10 | 54431 | |
| | | mRNA sequence (SEQ ID NO: 308): | |
| | | NM_018981.1 | |
| | | PRT sequence (SEQ ID NO: 309): | |
| | | NP_061854.1 | |
| ERp18 | TXNDC12, AGR1, | 51060 | |
| | ERp19 | mRNA sequence (SEQ ID NO: 310): | |
| | | NM_015913.2 | |
| | | PRT sequence (SEQ ID NO: 311): | |
| | | NP_056997.1 | |
| ERp27 | NA | 121506 | |
| | | mRNA sequence (SEQ ID NO: 312): | |
| | | NM_152321.2 | |
| | | PRT sequence (SEQ ID NO: 313): | |
| | | NP_689534.1 | |
| ERp29 | ERp28, ERp31, PDI- | 10961 | |
| | DB | mRNA sequences (SEQ ID NOS 314-315): | |
| | | NM_001034025.1 NM_006817.3 | |
| | | PRT sequences (SEQ ID NOS 316-317): | |
| | | NP_001029197.1 NP_006808.1 | |
| ERp44 | TXNDC4 | 23071 | |
| | | mRNA sequence (SEQ ID NO: 318): | |
| | | NM_015051.1 | |
| | | PRT sequence (SEQ ID NO: 319): | |
| | | NP_055866.1 | |
| ERp46 | TXNDC5 | 81567 | |
| | | mRNA sequences (SEQ ID NOS 320-321): | |
| | | NM_001145549.1 | |
| | | NM_030810.2 | |
| | | PRT sequences (SEQ ID NOS 322-323): | |
| | | NP_001139021.1 NP_110437.2 | |
| ERp57 | Grp58, PDIA3 | 2923 | Antibodies |
| | | mRNA sequence (SEQ ID NO: 324): | (SPA-585, |
| | | NM_005313.4 | SPA-725) |
| | | PRT sequence (SEQ ID NO: 325): | |
| | | NP_005304.3 | |
| ERp72 | PDIA4, ERp70 | 9601 | Antibody |
| | | mRNA sequence (SEQ ID NO: 326): | (SPS-720) |
| | | NM_004911.4 | |
| | | PRT sequence (SEQ ID NO: 327): | |
| | | NP_004902.1 | |
| P5 | PDIA6, ERP5, | 10130 | |
| | TXNDC7 | mRNA sequence (SEQ ID NO: 328): | |
| | | NM_005742.2 | |
| | | PRT sequence (SEQ ID NO: 329): | |
| | | NP_005733.1 | |
| PDI | PDIA1, P4HB | 5034 | |
| | | mRNA sequence (SEQ ID NO: 330): | |
| | | NM_000918.3 | |
| | | PRT sequence (SEQ ID NO: 331): | |
| | | NP_000909.2 | |
| PDILT | | 204474 | |
| | | mRNA sequence (SEQ ID NO: 332): | |
| | | NM_174924.1 | |
| | | PRT sequence (SEQ ID NO: 333): | |
| | | NP_777584.1 | |
| PDIP | PDIA2 | 64714 | |
| | | mRNA sequence (SEQ ID NO: 334): | |
| | | NM_006849.2 | |
| | | PRT sequence (SEQ ID NO: 335): | |
| | | NP_006840.2 | |
| PDIR | PDIA5 | 10954 | |
| | | mRNA sequence (SEQ ID NO: 336): | |
| | | NM_006810.3 | |
| | | PRT sequence (SEQ ID NO: 337): | |
| | | NP_006801.1 | |
| TMX | TXNDC1 | 81542 | |
| | | mRNA sequence (SEQ ID NO: 338): | |
| | | NM_030755.4 | |
| | | PRT sequence (SEQ ID NO: 339): | |
| | | NP_110382.3 | |
| TMX2 | TXNDC14 | 51075 | |
| | | mRNA sequences (SEQ ID NOS 340-341): | |
| | | NM_001144012.1 NM_015959.2 | |
| | | PRT sequences (SEQ ID NOS 342-343): | |
| | | NP_001137484.1 | |
| | | NP_057043.1 | |
| TMX3 | TXNDC10 | 54495 | |
| | | mRNA sequence (SEQ ID NO: 344): | |
| | | NM_019022.3 | |
| | | PRT sequence (SEQ ID NO: 345): | |
| | | NP_061895.3 | |
| TMX4 | TXNDC13 | 56255 | |
| | | mRNA sequence (SEQ ID NO: 346): | |
| | | NM_021156.2 | |
| | | PRT sequence (SEQ ID NO: 347): | |
| | | NP_066979.2 | |

For example, Hsp70 family of genes are particularly susceptible for heat induction. Promoter sequences such as the heat shock element has been identified as a heat responsive element in these proteins. HSP70 family of proteins are the most temperature sensitive and highly conserved of the HSPs. The HSP70s are ATP-binding proteins and demonstrate a 60-80% base identity among eukaryotic cells. There are at least four distinct proteins in the HSP70 group (HSP72, HSP73, HSP75, and HSP78). These HSPs are particularly useful in the embodiments of the invention that rely on heat shock by increased temperature treatment.

As used herein the term “animal” refers to any multi-cellular animal including mammalian and non-mammalian animals. Thus, the term “animal” encompasses any mammal and any non-mammal animal, non-limiting examples including, human, mouse, rabbit, monkey, pig, bird, amphibian (e.g. frog etc.), fish, and nematode (C. elegans etc.). The methods of the present invention can also be applied to other organisms than animals, such as plants.

As used herein the term “contacting” or “treating” refers to exposing the test model, cleavage staged embryo, or cells derived from a cleavage staged embryo, to a test compound or stress inducer, whether it be a compound or heat. Contacting/treating is intended to include both in vivo and in vitro methods. Contacting/treating a sensitized test animal or cleavage stage embryo with a test compound or stress inducer in vivo requires administration of the compound to the animal. Administration to the animal can be by any one or combination of a variety of methods (e.g., parenterally, enterally and/or topically), including systemic and local administration, e.g. oral, intravenous or intramuscular/tissue injection. Dosages can be easily be determined by those of skill in the art by using routine methods. An effective amount of a stress inducer may be determined by administering increasing levels of such compound, and determining a level at which there is an observed increase in level of heat shock protein encoding mRNA and/or protein and/or post-translantional modification of HSP locus or loci in the cells of interest, e.g., increased or decreased methylation of the promoter regions, i.e., a stress response. For in vivo administration the test compound should be in a suitable pharmaceutically acceptable carrier.

Contacting cleavage stage embryos, or cells arising from the embryos, in vitro can be performed by any means known to those of skill in the art, e.g. adding the test compound or stress inducer directly to the in vitro culture media. An effective amount of a stress inducer may be determined by exposing cells to increasing levels of such compound, and determining a level at which there is an observed increase in levels of heat shock protein mRNA. Dosages of test compounds can be varied. In some embodiments the stress inducer is heat shock. Heat shock can be achieved by raising the incubation temperature of the culture containing the cleavage stage embryos or cells.

As used herein the term “pharmaceutically acceptable carrier” refers to compositions, carriers, diluents and reagents, are used interchangeably and represent that the materials are capable of administration to or upon an animal without the production of undesirable physiological effects such as distress, gastric upset and the like. The preparation of a pharmacological composition, which can include pharmaceutically acceptable salts, that contains active ingredients dissolved or dispersed therein is well understood in the art and need not be limited based on formulation. Typically such compositions are prepared as injectable either as liquid solutions or suspensions, however, solid forms suitable for solution, or suspensions, in liquid prior to use can also be prepared. The preparation can also be emulsified or presented as a liposome composition. The active ingredient can be mixed with excipients which are pharmaceutically acceptable and compatible with the active ingredient and in amounts suitable for use for delivery of the stress inducer as described herein. Suitable excipients are, for example, water, saline, dextrose, glycerol, ethanol or the like and combinations thereof. In addition, if desired, the composition can contain minor amounts of auxiliary substances such as wetting or emulsifying agents, pH buffering agents and the like which enhance the effectiveness of the active ingredient.

Pharmaceutically acceptable salts include the acid addition salts (formed with the free amino groups of the polypeptide) that are formed with inorganic acids such as, for example, hydrochloric or phosphoric acids, or such organic acids as acetic, tartaric, mandelic and the like. Salts formed with the free carboxyl groups can also be derived from inorganic bases such as, for example, sodium, potassium, ammonium, calcium or ferric hydroxides, and such organic bases as isopropylamine, trimethylamine, 2-ethylamino ethanol, histidine, procaine and the like. Exemplary of liquid carriers are sterile aqueous solutions that contain no materials in addition to the active ingredients and water, or contain a buffer such as sodium phosphate at physiological pH value, physiological saline or both, such as phosphate-buffered saline. Still further, aqueous carriers can contain more than one buffer salt, as well as salts such as sodium and potassium chlorides, dextrose, polyethylene glycol and other solutes. Liquid compositions can also contain liquid phases in addition to and to the exclusion of water. Exemplary of such additional liquid phases are glycerin, vegetable oils such as cottonseed oil, and water-oil emulsions. For topical administration, creams, ointments, jellies, solutions or suspensions, etc. can be used.

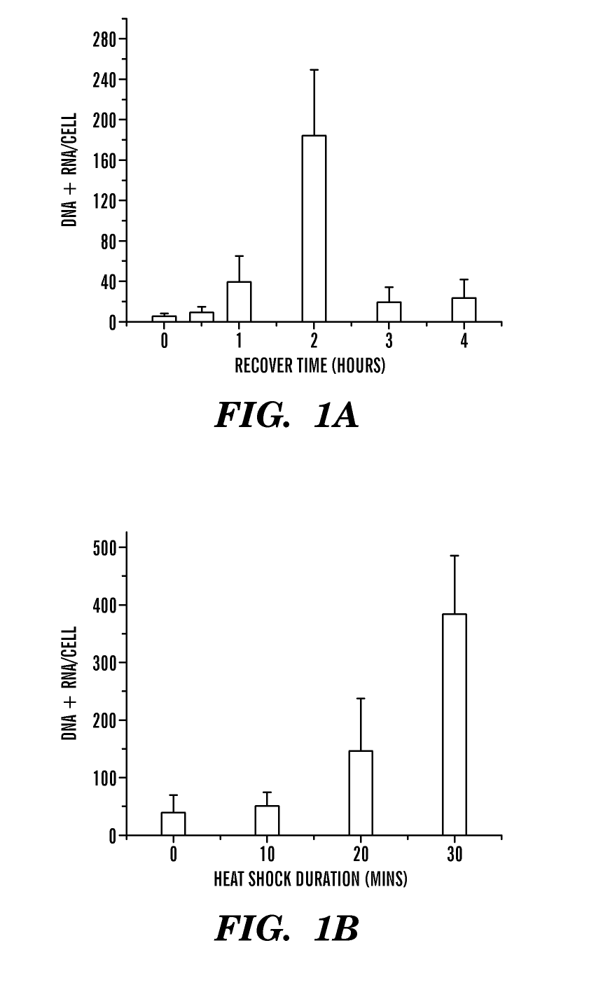

The phrase “a greater response to a second treatment with a stress inducer” as used herein refers to a statistically significant increase in expression level (e.g. statistically significant increase in measured protein or mRNA levels) of a heat shock protein in response to a secondary treatment with a stress inducer as compared to the measured expression levels of the heat shock protein after treatment with a primary stress inducer. For comparison, the levels of heat shock protein should be assessed at the same time point after the primary and secondary treatment, respectively. It is preferred that the comparison be performed at a time point after treatment that results in the maximum level of heat shock protein. One of skill in the art understands that this time point may vary between animals, species and cells, however the time point of maximum expression can be easily determined experimentally (e.g., see Example 1 and FIG. 1a).

Embodiments of the invention encompass methods for making embryos and embryonic cells arising from them, that are sensitized to stress, e.g. physiologic or chemical stressors.

In one embodiment the sensitized embryos and embryonic cells arising from them are made by treating a cleavage stage embryo or cell with a stress inducer such that the levels of a heat shock protein or mRNA are increased to a level that renders the embryo or cell more sensitive to a secondary treatment with a stress inducer.

The dose of stress inducer (heat or chemical or other stress) required to increase the levels of heat shock protein (HSP) or HSP mRNA to a sufficient level such that the embryo or cells exhibit a greater response to a second treatment with a stress inducer (i.e. exhibit heat shock “memory”) can be assessed experimentally. For example, by performing a series of primary and secondary treatments with a stress inducer at varied doses and time of exposure (the dose test system) and using as a control, cleavage stage embryos or cells arising from the cleavage stage embryos that have received only one treatment of stress inducer given at the same developmental stage and at the same dose as the secondary treatment in the dose test system. The secondary treatment is given only after HSP mRNA levels return to normal subsequent to any primary treatment. In one embodiment, the primary and secondary doses are the same. The level of heat shock protein in the embryo or cells after the secondary treatment, and the level of heat shock protein in the control after the individual stress inducer treatment can then be assessed. If the level of heat shock protein in the dose test system is significantly increased as compared to the level in the control, then the respective dose of stress inducer increases the levels of heat shock protein to a sufficient level to render the embryo/cell more responsive to a stress inducer, i.e. to exhibit a greater response to a second treatment with a stress inducer. The time of exposure to a stress inducer in order to achieve heat shock “memory” can vary depending on the particular stress inducer used in methods of the invention, and can easily be determined by those of skill in the art. To the extent that there are variations between animals and species, correct dosages can easily be determined experimentally by those of skill in the art, e.g., by starting with defined dosages.

Induction of Hsp70 has been previously associated with the development of tolerance to a variety of stresses, heat, including hypoxia, ischemia, acidosis, energy depletion, cytokines such as tumor necrosis factor-(TNF-), and ultraviolet radiation. Accordingly, any of these various stressors can be used in the present methods to sensitize the embryos or embryonic stem cells to stress.

In one embodiment the stress inducer is heat shock applied in vitro by raising the incubation temperature of the culture containing the cleavage stage embryo or cells arising from the cleavage stage embryo. For example the temperature can be raised from 37° C. to 43° C. The length of time that the cleavage stage embryo or cells arising from the cleavage stage embryo is exposed to the increased temperature can vary. In one embodiment, the exposure to heat shock is from about 10 minutes to about 25 minutes.

As used herein, “an increased level of HSP protein” refers to an amount of HSP protein or HSP mRNA that is greater, by a statistically significant amount, than the amount of protein or mRNA present in a comparison sample, e.g. a control sample. In one embodiment the increased level is at least 2-fold greater. It should be understood that the levels of HSP can be represented by arbitrary units, for example as units obtained from a densitometer, luminometer, a Fluorescence Activated Cell Sorting (FACS) machine or an ELISA (Enzyme-Linked ImmunoSorbent Assay), plate reader.

The term “statistically significant” or “significantly” refers to statistical significance and generally means a two standard deviation (2SD) above or below normal or control levels.

Heat shock proteins (HSPs) are expressed in virtually all cells in response to environment stressor and serve to protect the cells from heating and toxic substances, which might occur during embryo culture¹³⁻¹⁵. Three major groups of HSPs have been described, HSP70, HSP90 and HSP110¹³, plus numerous small HSPs¹⁶. The phenomenon of thermotolerance, a first mild heat exposure of the embryo protecting it from a second more severe heat shock, has been observed in post-implantation mouse embryos¹⁷⁻¹⁹. It was suggested that HSP70 has a direct role in the induction of thermotolerance and that the level of thermotolerance can be correlated to the level of HSP70 protein¹⁸. Although inducible expression of the hsp70i genes (hsp70.1 and hsp70.3) is observed in mouse embryos starting at the 4-cell stage³, the relationship between hsp70i gene expression and thermotolerance in preimplantation embryos is unclear.

The levels of any heat shock protein (HSP) that responds to a stressor can be monitored. In some embodiments, HSP70 family of proteins are monitored to detect the heat shock response in response to treatment with increased temperature. In some embodiments, the level of Hsp70i is assessed. Other examples of heat shock proteins that can be monitored include, but are not limited to, Hsp10, Hsp27, HspB1, Hsp40, Hsp60, Hsp71, Hsp70, Hsp72, Grp78 (BiP), Hsx70, Hsp70i, Hsp90, Grp94, Hsp104, and Hsp110.

Table 1 sets forth an exemplary list of heat shock proteins (HSPs) any one or more of which can be used in monitoring a heat shock response of a cell according to the methods described herein. For example, based on the provided information, a skilled artisan can readily design primers to amplify any one or more of the listed HSPs or to detect changes in RNA expression levels. A skilled artisan may also use the proteins to make antibodies using usual immunization protocols well known to one skilled in the art. Such antibodies can be used to detect the level of protein expression of one or more of the HSPs in a cell.

HSP levels can be assessed by any variety of means known to those of skill in the art. One can detect levels of HSP transcripts, levels of HSP proteins, and the level of posttranslational modification of various HSPs and their promoter regions.

In some embodiments the level of HSP is assessed by measuring the level of HSP protein. Means for measuring the levels of protein are well known to those of skill in the art.

In one embodiment an immunoassay is used. Anti-HSP antibodies are commercially available, and some examples of these are listed in Table 1. In particular, antibody-based detection techniques such as immunohistochemistry, immunocytochemistry, FACS scanning, immunoblotting, radioimmunoassays, western blotting, immunoprecipitation, enzyme-linked immunosorbant assays (ELISA), sandwich ELISA, and derivative techniques that make use of antibodies directed against activated heat shock proteins can be used.

Antibodies, polyclonal or monoclonal, can be purchased from a variety of commercial suppliers, or may be manufactured using well-known methods, e.g., as described in Harlow et al., Antibodies: A Laboratory Manual, 2nd Ed; Cold Spring Harbor Laboratory Press, Cold Spring Harbor, N.Y. (1988). In general, examples of antibodies useful in the present invention include anti-heat shock protein antibodies. Such antibodies can be purchased, for example, from Upstate Biotechnology (Lake Placid, N.Y.), New England Biolabs (Beverly, Mass.), and NeoMarkers (Fremont, Calif.).

Alternative methods for measuring protein levels include Mass Spectrometry such as MALDI/TOF (time-of-flight), SELDI/TOF, liquid chromatography-mass spectrometry (LC-MS), gas chromatography-mass spectrometry (GC-MS), high performance liquid chromatography-mass spectrometry (HPLC-MS), capillary electrophoresis-mass spectrometry, nuclear magnetic resonance spectrometry, or tandem mass spectrometry (e.g., MS/MS, MS/MS/MS, ESI-MS/MS, etc.). See for example, U.S. Patent Application Nos: 20030199001, 20030134304, 20030077616, which are herein incorporated by reference.

In one embodiment the levels of heat shock protein (HSP) are assessed by measuring the level of mRNA in the embryo or cells. Means for measuring mRNA are well known to those of skill in the art and any suitable means can be used. In one embodiment, the levels of mRNA are assessed by using real-time one-step RT-LATE-PCR, for example as described in Example 1. An alternative means for assessing the level of HSP mRNA includes the detection of RNA transcripts by Northern blotting, for example, wherein a preparation of RNA is run on a denaturing agarose gel, and transferred to a suitable support, such as activated cellulose, nitrocellulose or glass or nylon membranes. Radiolabeled cDNA or RNA is then hybridized to the preparation, washed and analyzed by autoradiography. Detection of RNA transcripts can further be accomplished using other known amplification methods which include, but are not limited to, the so-called “NASBA” or “3SR” technique described in PNAS USA 87: 1874-1878 (1990) and also described in Nature 350 (No. 6313): 91-92 (1991); Q-beta amplification as described in published European Patent Application (EPA) No. 4544610; strand displacement amplification (as described in G. T. Walker et al., Clin. Chem. 42: 9-13 (1996) and European Patent Application No. 684315); and target mediated amplification, as described, e.g., in PCT Publication WO 9322461.

Alternatively, mRNA expression can be detected on a DNA array, chip or a microarray. Oligonucleotides corresponding to the heat shock protein are immobilized on a chip which is then hybridized with labeled nucleic acids of a test sample obtained from a patient. Positive hybridization signal is obtained with the sample containing heat shock protein transcripts. Methods of preparing DNA arrays and their use are well known in the art. (See, for example U.S. Pat. Nos. 6,618,6796; 6,379,897; 6,664,377; 6,451,536; 548,257; U.S. 20030157485 and Schena et al. 1995 Science 20:467-470; Gerhold et al. 1999 Trends in Biochem. Sci. 24, 168-173; and Lennon et al. 2000 Drug discovery Today 5: 59-65, which are herein incorporated by reference in their entirety). Serial Analysis of Gene Expression (SAGE) can also be performed (See for example U.S. Patent Application 20030215858). To monitor mRNA levels, for example, mRNA is extracted from the biological sample to be tested (animal tissue, embryos, or cells from embryo), reverse transcribed, and fluorescent-labeled cDNA probes are generated. The microarrays capable of hybridizing to heat shock protein cDNA are then probed with the labeled cDNA probes, the slides scanned and fluorescence intensity measured. This intensity correlates with the hybridization intensity and expression levels.

In one embodiment the levels of HSP are monitored by measuring the level of a visibly detectable protein that is operably linked to a HSP promoter. For example, cell strains and mice can be generated in which a gene for an easily visualized protein, such as green fluorescent protein, or one of its derivatives, is under the control of one or more members of the heat shock genes. In this manner, the response to a stress inducer can be monitored by visualizing the protein.

Posttranslational modifications, such as methylation, ubiquitination, phosphorylation and sumoylation, of HSP encoding genes can also be monitored using techniques known to one skilled in the art. Methods such as those described in U.S. Patent Application Publication Nos. 20090087919 and 20100160177 can be used to determine the level of posttranslational modification before and after contacting the embryos or cells with a stress inducer.

In one embodiment the stress inducers are added in vitro. Chemical stress inducers can be added directly to the culture media containing the cleavage stage embryo, or cells arising from the embryo. After addition of the chemical stress inducer as a primary treatment, for a given period of time, the cells/embryo are washed, typically extensively to remove the inducer form the culture.

Any number of cleavage stage embryonic cells can be treated. In one embodiment, 1-32 embryonic cells are treated.

When heat shock is used as a stress inducer, the heat shock can be applied in vitro by raising the incubation temperature of the culture. In one embodiment the mammalian embryos/cells are treated with a heat shock ranging from about 40° C. to about 45° C. usually in a time frame of about 10 to about 25 minutes.

In some embodiments, the stressor, such as heat shock or chemical exposure times can be 10-20 minutes, 10-15 minutes, 15-20 minutes, 15-25 minutes, or 20-25 minutes. In some embodiments, the stressor, such as a heat shock is applied for 5 or 10 to up to 30 minutes. In one embodiment, mammalian embryos/cells are treated with a heat shock of 43° C. for 20 minutes.

When the stress inducer is a chemical, the treatment time often varies from minutes to hours. For example from 30 minutes to 45 minutes, 50 minutes, or about 1, 2, 3, 4, 5, or 6 hours. The treatment time can be regulated by transferring the embryo or cell from a culture medium to which the stress inducer has been added to a culture medium without the stress inducer. The cells may be washed during the transfer process.

“Heat shock” as used herein generally refers to a temperature that is raised 4-6° C. above an optimal cell growth temperature. The optimum temperature range for heat shock protein induction ranges considerably with the organism but relates to the physiological range of supraoptimal temperatures within which active adaptation is observed. For example, this is typically around 40-50° C. for birds and mammals, 35-37° C. for Drosophila, 33-35° C. for yeast, and 35-40° C. for plants (Burdon, Biochem J. 240: 313-324, 1986).

In some embodiments, a heat shock with increased temperature is induced in cells by increasing the cell or embryo culture temperature by 2° C., 3° C., 4° C., 5° C., 6° C., 7° C., 8° C., 9° C. or 10° C. compared to the normal or optimal cell culture temperature. In some embodiments, the temperature increase can be 1.5, 2.5, 3.5, 4.5, 5.5, 6.5, 7.5, 8.5 or 9.5° C. In some embodiments a temperature increase between about 2-10° C., 2-8° C., 2-6° C., 2-4° C., 2-3° C., 4-10° C., 4-8° C., and 4-6° C., and any increments between these temperatures can be applied.

A skilled artisan is readily able to determine the normal or optimal temperatures for growing or culturing various types of embryos, cells or embryonic stem cells from well established literature for cell growth conditions.

For example, typical normal or optimal temperature for growing or culturing mammalian cells, embryos or embryonic cells is 37° C. or about 37° C., and often the temperature is kept at 37±0.5° C. However, while adult fish, larvae, and embryos remain viable over a wide range of core body temperatures (temperature range can be as high as 10° C.), the optimal temperature for growing cells from frogs, such as Xenopus, and fish, such a gold fish or zebra fish, Danio rerio, is often at room temperature (about 22° C. to about 25° C.), for example 25° C. (a typical zebrafish cell growth temperature), 23° C. or about 23° C. (a typical gold fish cell growth temperature), 22° C. (a typical Xenopus cell growth temperature).

In some embodiments, sensitized embryos or embryonic cells are made by treating a cleavage stage embryo or embryonic cell twice with a stress inducer, i.e. a first and second treatment. After the first, primary treatment, the cleavage stage embryo or embryonic cells should be given sufficient time to recover prior to the second treatment, i.e. the levels of HSP should return to normal (i.e. expression levels present in the absence of a primary treatment) before the second treatment is applied.

Both the first and the second treatment can be applied in vitro or in vivo. Alternatively for a cleavage stage mammalian embryo the primary treatment can be applied in utero, and after extraction of the embryo from the uterus the secondary treatment applied in vitro; or the primary treatment can be applied in vitro and after implantation, the secondary treatment applied in utero. Heat shock treatments are performed in vitro.

Methods for making test animals sensitized for stress are also provided.

In one aspect, methods for making sensitized test non-mammalian animals is provided. The methods involve i) treating a cleavage staged embryo with a stress inducer such that the levels of a heat shock protein or mRNA are increased to a sufficient level to exhibit a greater response to a second treatment with a stress inducer; ii) developing the treated cleavage staged embryo to an advanced stage of development and multi-cellular differentiation, whereby the multi-cellular animal exhibits a sensitized response to a secondary stress inducer. In one embodiment, a second treatment is performed in vitro at a later stage of development, i.e. a stage of development after said first treatment. Developmental stages of non-mammalian embryos are well known to those of skill in the art and can be found in, for example “Non-mammalian animal models for biomedical research” by Avril D. Woodhead, CRC Press, Brookhaven National Lab, Upton, N.Y., © 1989; “Developmental Biology” sixth edition, by Scott F. Gilbert, Sinauer Associates Inc, © 2000. As used herein, an “advanced stage of development” refers to a stage occurring after the cleavage stages of development, e.g. after blastula formation.

In some embodiments, the first and the secondary stress inducers are the same, such as heat, or anoxia, or the same chemical.

In some embodiments, the first and the second stress inducers are different, such as a first chemical and a second, different chemical, or heat first and a chemical second, or heat first and anoxia second, or a chemical first and heat second, or a chemical first and anoxia second, or any other combination of the various stress inducers known to one skilled in the art.

In one aspect, methods for making sensitized test mammals or test plants are provided. The methods involve treating a cleavage staged mammalian embryo or an embryo of a corresponding stage from plants, with a stress inducer such that the levels of a heat shock protein or mRNA are increased to a sufficient level to exhibit a greater response to a second treatment with a stress inducer and then implanting the treated embryo into the uterus of a receptive female for in-vivo growth and birth, or, if plant, culturing the embryo in a plant embryo culture to allow it to develop into a seed, where the birthed animal, or the plant seed and ultimately the grown plant exhibits a sensitized response to a secondary stress inducer.

The dosage of stress inducer to be applied, such that the levels of a heat shock protein or mRNA are increased to a sufficient level to exhibit a greater response to a second treatment with a stress inducer, can be determined by performing a series of primary and secondary treatments with a stress inducer at varied doses and time of exposure (the dose test system) and using as a control, cleavage stage embryos or cells arising from the cleavage stage embryos that have received only one treatment of stress inducer given at the same developmental stage and at the same dose as the secondary treatment in the dose test system. The secondary treatment is given only after the HSP mRNA levels return to normal subsequent to any primary treatment. In one embodiment, the primary and secondary doses are the same. The level of heat shock protein in the embryo or cells after the secondary treatment, and the level of heat shock protein in the control after the individual stress inducer treatment can then be assessed. If the level of heat shock protein in the dose test system is significantly increased as compared to the level in the control, then the respective dose of stress inducer increases the levels of heat shock protein to a sufficient level to render the embryo/cell more responsive to a stress inducer, i.e. to exhibit a greater response to a second treatment with a stress inducer.

Means for culturing and implantation of mammalian embryos are well known to those of skill in the art, for example methods can be found in Manipulating the Mouse Embryo: A Laboratory Manual (Second Edition), by Brigid Hogan, Vanderbilt University Medical School; Rosa Beddington, National Institute for Medical Research, London; Frank Costantini, Columbia University; Elizabeth Lacey, Memorial Sloan-Kettering Cancer Center© 1994•487 pp; and the book entitled “Nuclear Transfer Protocols: Cell Reprogramming and Transgenesis Series: Methods in Molecular Biology”, Volume: 348, Pub. Date: Jul.-18-2006.

The embryos can be transferred back to uterus after stress treatment. For example in human in vitro fertilization (IVF) protocols, embryos have traditionally been transferred into the uterus after only 2 or 3 days of culture. In some protocols, they can be grown to a blastocyst level prior to transplantation to select for the most viable embryos for further transplantation. Blastocyst is an embryo that has developed for five to seven days after fertilization and has developed two distinct cell types and a central cavity filled with fluid (blastocoel cavity). The cells in a blastocyst have just started to differentiate. The surface cells that surround the cavity (just under the outer shell) are called the trophectoderm and will later develop into the placenta. The more centrally located group of cells are called the inner cell mass and will become the fetus.

The timing of implantation into uterus can be optimized based on the animal's natural embryonic development. So, for example in human, the embryo arrives into uterus about 80 hours after fertilization and the implantation into the lining of the uterus typically occurs at around day 5. Thus, it has been postulated that the lining of the uterus is most receptive for the embryo to implant into it on day 5 resulting in better rates of IVF success. Similarly, the most optimal timing of implantation can be determined for any animal.

In plants, embryogenesis occurs naturally as a result of sexual fertilization and the formation of the zygotic embryos. The embryo along with other cells from the motherplant develops into the seed or the next generation, which, after germination, grows into a new plant. The plant embryos can be treated using the stress inducers in a similar manner as the animal cells. Treated embryos from plants can then be cultured using well established techniques (see, e.g., S. Narayanaswami and Knut Norstog, Plant embryo culture, The Botanical Review, Volume 30, Number 4/October, 1964, 2008).

In one aspect, to make the test mammalian animal sensitized for stress, the cleavage stage embryo from an animal or a corresponding stage of a plant embryo is treated with a primary stress inducer and a secondary treatment is also performed. The secondary treatment with a stress inducer can be preformed in vitro prior to implantation or in utero after implantation.

Methods for screening for compounds that inhibit the induction of stress by a stress inducer using a test model selected from a sensitized test animal/test plant, or a sensitized embryo, or embryonic cell, of the invention are also provided. These model systems are amenable to high throughput screenings. For example, in one embodiment, sensitized embryonic cells are tested in vitro in multi-well tissue culture plates.

The methods involve i) contacting a test model with a test compound ii) contacting the test model with a stress inducer, ii) assessing the level of heat shock protein or mRNA in cells of the test model, and iii) comparing the level of heat shock protein or mRNA determined to a control level. The control level is a level of heat shock protein or mRNA observed in cells of a corresponding test model treated with the same stress inducer and dose as the test model, but not treated with the test compound. A decreased level of heat shock protein or mRNA as compared to the control level indicates that the test compound is an inhibitor of the stress inducer.

A decreased level of heat shock protein (HSP) refers to an amount of HSP protein or HSP mRNA that is reduced in comparison to the amount of protein or mRNA present in the control sample. The reduction is considered valid if it is statistically significant. In some embodiments, the reduction is for example about 50-75% or 50-80%. In some embodiments the decreased level is at least 2 fold less.

The invention also encompasses methods for screening for compounds that induce stress. The methods involve i) contacting a test model selected from a sensitized test animal, or a sensitized embryo or embryonic cell of the invention with a test compound, then contacting the test model with a stress inducer, ii) assessing the level of heat shock protein or mRNA in cells of the test model; and iv) comparing the level of heat shock protein or mRNA determined to a control level. The control level is a level of heat shock protein or mRNA observed in cells of a corresponding test model treated with the same stress inducer as the test model, but not treated with the test compound. An increased level of heat shock protein or mRNA in the test model as compared to the control level indicates that the test compound is an inducer of stress.

An increased level of HSP protein refers to an amount of HSP protein or HSP mRNA that is greater than the amount of protein present in a control sample. The increase is statistically significant. In one embodiment the increased level is at least 2 fold greater.

It should be understood that the levels of HSP can be represented by arbitrary units, for example as units obtained from a densitometer, luminometer, a Fluorescence Activated Cell Sorting (FACS) machine or an ELISA (Enzyme-Linked ImmunoSorbent Assay) plate reader. Alternatively, readings from real time PCT systems, and other nucleic acid relative or absolute quantification systems, such as MALDI-TOF, may also be used.

In one embodiment, the screen is performed with test model systems that comprise a reporter gene operably linked to regulatory sequences of a heat shock protein. As such, treatment with a stress inducer induces expression of the reporter gene and the level of reporter gene can be measured. Heat shock promoters have extensively been used in different experimental systems, and are well known to those of skill in the art. The highly conserved nature of the heat stress response allows the use of heterologous promoters.

Heat shock protein regulatory sequences for the purposes of the systems of the invention include but are not limited to heat shock element of HSP70. One of the most conserved heat responsive elements in the HSP encoding genes is a heat shock element. It is well conserved among eukaryotic cells with a sequence of CT-GAA-TTC-AG, and is also called the “Pelham box”. It is located on the promoter region of the HSP encoding genes upstream of the TATA box.

As used herein, the term “operably linked” means that the regulatory sequences necessary for expression of the coding sequence of a reporter gene are placed in the DNA molecule in the appropriate positions relative to the coding sequence so as to effect expression of the coding sequence. This same definition is sometimes applied to the arrangement of coding sequences and transcription control elements (e.g. promoters, enhancers, and termination elements) in an expression vector. This definition is also sometimes applied to the arrangement of nucleic acid sequences of a first and a second nucleic acid molecule wherein a hybrid nucleic acid molecule is generated.

As used herein, the term “reporter gene” refers to a gene that encodes a product which when expressed produces a reporter signal, a reporter gene product, that is readily measurable, e.g., by biological assay, immunoassay, radioimmunoassay, or by colorimetric, fluorogenic, chemiluminescent or other method.

Suitable reporter genes for use in methods of the invention include, but are not limited to, LacZ, green fluorescent protein (GFP); green fluorescent-like protein (GFP-like); yellow fluorescent protein (YFP); blue fluorescent protein (BFP); enhanced green fluorescent protein (EGFP); enhanced blue fluorescent protein (EBFP); cyan fluorescent protein (CFP); enhanced cyan fluorescent protein (ECFP); red fluorescent protein (dsRED); and modifications and fluorescent fragments thereof.

In methods of the invention, contacting a sensitized test animal, a sensitized embryo, or sensitized embryonic cell of the invention with a test compound, or a stress inducer, can be performed in vitro or in vivo. In vitro by adding the test compound or chemical stress inducer directly to the culture media for culturing of embryos and embryonic cells. In vivo, by use of a pharmaceutically acceptable carrier.

The invention will be further illustrated by the following non-limiting examples.

### EXAMPLES

### Example 1

### Heat Shock “Memory” Renders Embryos More Sensitive to Secondary Stressors

To investigate whether brief heat treatment of preimplantation cleavage-stage mouse embryo can alter the normal pattern of gene expression, or render the blastocyst stage embryo either thermotolerant or more sensitive to secondary heat treatment, we measured hsp70i mRNA levels in single cells isolated from 8-cell stage mouse embryos that had been treated one or more times with different doses of heat.

Levels of hsp70i mRNA per cell in 8-cell stage embryos heated for 20 minutes at 43° C. reached maximum levels two hours after treatment and then returned to normal after four hours (FIG. 1a). A heating period of only 10 minutes did not result in elevated hsp70i mRNA after two hours of recovery, while the average number of hsp70i mRNA+DNA molecules per cell following 20 or 30 minutes of heating was found to be, respectively, 145 (±92) and 383 (±100). Thus, levels of hsp70i mRNA gene expression correlate with the length of heat shock (FIG. 1b).

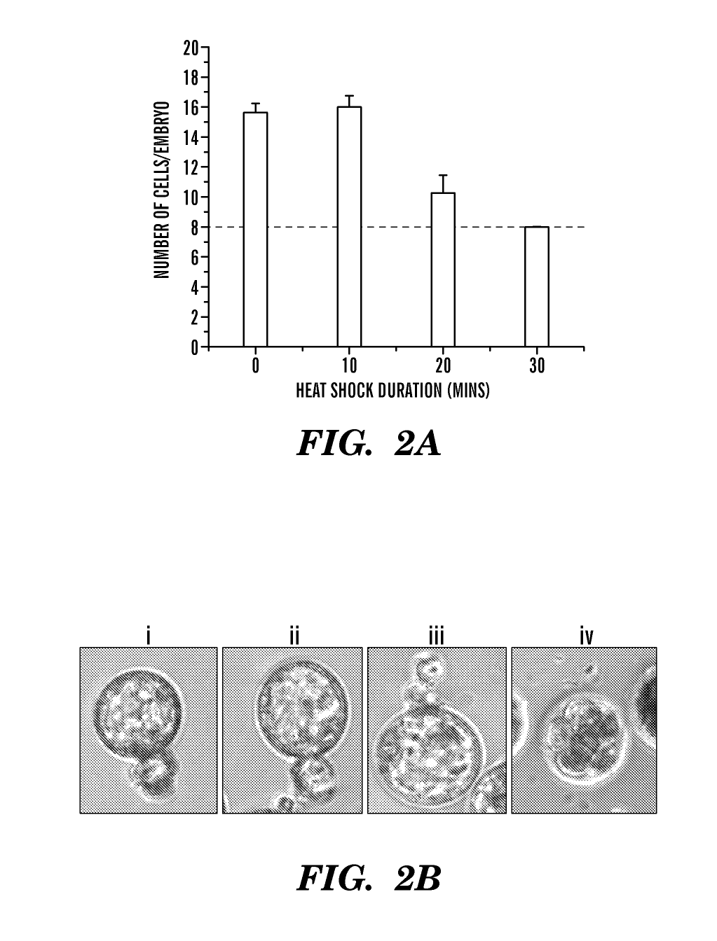

The fact that longer heating results in more heat shock hsp70i mRNA accumulation does not, however, guarantee that embryos treated for 20 or 30 minutes are equally able to recover from the stress. FIG. 2 shows that embryos heated for 30 minutes at the 8-cell stage and then cultured for an additional 24-48 hours at 37° C. failed to undergo cell division and eventually died. In contrast, embryos heated for 10 minutes continued to grow just like control embryos, while embryos treated with heat for 20 minutes paused cell division for several hours (FIG. 2) and then resumed growth and development to blastocyst stage as measured by viewing embryo morphologies 48 hours after heat shock at the 8-cell stage for different lengths of time [i) no heat shock; ii) heat-shocked for 10 minutes; iii) heat-shocked for 20 minutes; iv) heat-shocked for 30 minutes] (data not shown). Thus, although the time needed to initially reach blastocyst stage was not the same for untreated embryos and embryos heated for 20 minutes, both sets of embryos were morphological similar after 48 hours, with a well formed blastocoel having an inner cell mass on one side (data not shown). Both sets of embryos also went on to hatch data not shown).

In order to study preimplantation embryos' response to double heat shock, we exposed 8-cell stage embryos to 20 minutes of heat and then, after a gap of 4 hours, tested them for thermotolerance by heating them again for either 10 or 30 minutes. Primary heat treatment for 20 minutes did not prevent developmental arrest caused by a higher dose of heat (data not shown), demonstrating that these embryos were not rendered thermotolerant, even though they exhibited a robust response to the first heat treatment. To determine this embryos were heat shocked at the 8-cell stage either a) embryo heat-shocked for 20 minutes and then again for 10 minutes after a 4-hour recovery period or b) embryo heat-shocked for 20 minutes and then again for 30 minutes after a 4-hour recovery period. The embryos were then cultured for an additional 44 hours, when micrographs were taken (data not shown).

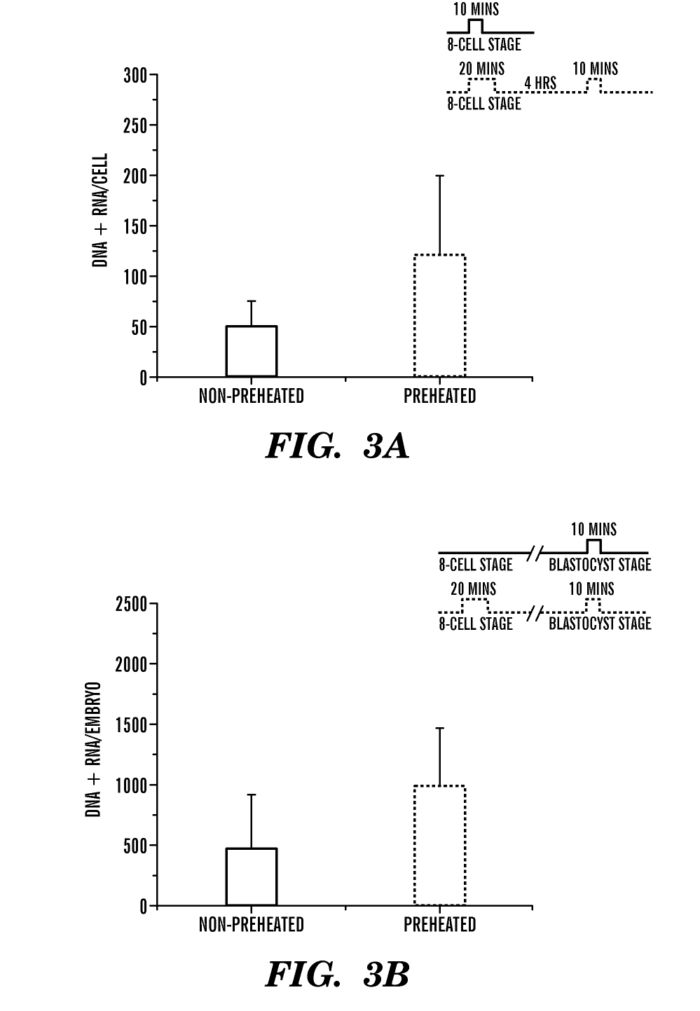

We next investigated whether a primary exposure to heat could, in fact, sensitize the embryo to secondary heat treatment. One group of 8-cell stage embryos was heat-treated for 20 minutes, allowed to recover for 4 hours, and then heated again for 10 minutes. The level of hsp70i mRNA in the cells of these embryos was compared to that of embryos which were heated only once for 10 minutes. Because the embryos paused in their development following the 20 minutes heat shock (FIG. 2a), both groups of embryos were at the 8-cell stage during all phases of this experiment. The results in FIG. 3a show that single cells from embryos heated only once contained an average of 50 (±25) copies of hsp70i mRNA+DNA. In contrast, double heat-shocked embryos had an average of 122 (±78) copies of mRNA+DNA per cell, demonstrating that the initial 20-minute treatment sensitized these cells to secondary heating. We call this phenomenon heat shock “memory” in mouse preimplantation embryos.

The fact that the sensitized embryos in the above experiment were always at the 8-cell stage made us wonder whether heat shock memory would persist through one or more rounds of DNA replication and cell division. To test this possibility we once again heated a group of about twenty 8-cell embryos for 20 minutes and then grew them to the blastocyst stage. At this point the embryos were heated a second time for just 10 minutes. A second group of about twenty control embryos was not heated at the 8-cell stage, but were exposed to heat for 10 minutes when they reached the blastocyst stage. Because single cells cannot readily be dissected out of blastocyst stage embryos, we analyzed whole embryos and measured the hsp70i mRNA+DNA copy numbers two hours after the 10-minute heat treatment.

As shown in FIG. 3b, the double heat-shocked embryos contained on the average 984 (±478) copies of hsp70i templates per embryo while the embryos heat-shocked once yielded only 476 (±444) copies of hsp70i templates per embryo, a ratio of about 2:1. The ratio of hsp70i mRNA molecules per cell in the pretreated embryos versus control embryos, however, is likely to be significantly higher because the primary exposure to heat arrests cell division for several hours. Therefore, the preheated blastocysts contain fewer cells, on average, than the control blastocysts. The higher response in the blastocyts first heat-treated at the 8-cell stage demonstrates that heat shock “memory” persists through several cell divisions.

Given the heat shock “memory” that is observed with primary treatment of embryos, as indicated by the increased level of hsp70i mRNA upon secondary treatment, we expect that other members of the heat shock protein family will exhibit heat shock “memory” in cleavage-stage embryos as well. Furthermore, since stressors other than heat, e.g. chemicals, are known to activate heat shock gene expression, we expect that a wide range of chemicals, including amino acid analogs, □-difluoromethylornithine^{20,21}, household chemicals and environmental pollutants are stressors affected by heat shock “memory”, i.e. embryos and embryonic cells will be more sensitive to such stressors after a primary insult, whether it is from the same stressor or different one. Such stressors could affect a cleavage-stage human embryo in utero or in vitro in an IVF clinic.

We are investigating why 8-cell embryos given 20 minutes of primary heat treatment pause in cell division and how they resume cell division. Kiessling et al.²⁹ has recently shown that normal human embryos at the 8-cell stage have elevated levels of mRNAs for cell cycle progression and the circadian rhythm, while mRNAs for cell cycle checkpoints are absent. We anticipate that our tests in mouse embryos will show that primary heat treatment leads to the loss of the mRNAs for cell cycle progress, but they accumulate again before the 8-cell embryo resumes dividing.

The presence of heat shock “memory” may significantly change the way that we approach in vitro fertilization (IVF) and think about the susceptibility of in utero cleavage stage embryos to stress. The presence of heat shock “memory” in blastocysts after primary treatment of a pre-implantation cleavage-stage embryo in the 8-cell stage provides evidence that heat shock “memory” will be stable in at least some cells derived from the embryo during fetal and neonatal development and thus result in increased sensitization to secondary stressors in these cells (heat, chemicals, environmental pollutants, and household chemicals).

Generation of Mouse Test Models for Screening of Stressors and their Inhibitors

Heat shock memory is very useful for screening chemicals and drugs for their possible effects as secondary inducers of stress, as well as their capacity to down regulate or up-regulate secondary induction of physiological stress. In order to make it easier to detect and quantify heat shock memory in one or more cell types in an implanted embryo, fetus, neonate, or developing animal we are using mice as a model organism. We are constructing strains of mice in which the gene for an easily visualized protein, such as the green fluorescent protein (or one of its derivatives) is under control of one or more members of the heat shock genes, shown in the experiments above to generate a robust response to primary treatment with heat or a chemical. Using these constructs we expect that prior to stimulation the cells of the embryo will exhibit little or no expression of the fusion gene. Following primary stimulation the mRNA of the fusion gene will accumulate over two hours, the protein will be expressed and the cells will transiently become fluorescent. Later when the cells or even the entire organism is induced for heat shock a second time, one or more cell types in the developing or adult animal will again become fluorescent. This secondary response to heat shock will be brighter and/or more rapid than the equivalent response in an animal that has not been treated once. Heat shock memory will be investigated and quantified at the RNA level by using LATE-PCR assays and multiplexed assays for members of the heat shock family of genes. Each cell type will be tested on its own after primary induction of heat shock in vivo with a chemical or in vitro with either a chemical stimulus or heat treatment, followed by secondary stimulation in vitro.

### Methods

2-cell stage mouse embryos from Embryotech Laboratories, Inc were cultured as described elsewhere²³. Heat shock was performed at 43° C. and the embryos were allowed 2-hours recovery time at 37° C. before harvesting. Single cells were isolated from 8-cell stage embryos through a hole in the zona pellucida ablated using a ZILOS-tk™ laser optical system²⁴. PurAmp single-tube method²⁵ was used for lysing single cells or whole embryos and for analyzing them by real-time one-step RT-LATE-PCR^{26,27} in the same vessel. The average number of hsp70i mRNA+DNA copies per cell was obtained using 30-50 single cells from each group of embryos. RT-PCR was run in 50 μl reaction with 50 nM limiting primer, 5′-CAGCGTCCTCTTGGCCCTCTCACAC-3′ (SEQ ID NO:1), 2 μM excess primer, 5′-GATCGACGACGGCATCTTC-3′ (SEQ ID NO:2), 500 nM. Probe, BHQ1-5′-GATCCTCTTGAACTCCTTC-3 (SEQ ID NO:3)-Cal Orange 560. (BHQ1: Black Hole Quencher 1) along with 300 nM Primesafe I™ (Smith Detection, UK)²⁸, and 1 μl of SSIII/Platinum Taq mixture (Invitrogen, 11732) in standard PCR buffer (Invitrogen, 11304). The cycling profile was: 55° C. for 15 minutes (RT); 95° C. for 5 minutes; 15 cycles consisting of the following three steps: 95° C. (10 sec), 63° C. (20 sec) and 72° C. (30 sec); 35 cycles with the following four steps: 95° C. (15 sec), 55° C. (25 sec), 72° C. (35 sec) and 45° C. (30 sec) (Fluorescence reading). Standard curves for template copy number quantification were generated with the same pair of primers, using a template of purified mouse genomic DNA (Sigma, D4416) in serial dilutions containing the following number of copies: 1,000, 100, 10 and 1. The C_{T} values obtained from real-time LATE-PCR of experimental samples were then converted into copy number by using these standards. Standards were included in each experiment to account for some batch-to-batch reagents variability. The quantitative accuracy of the one-step RT-LATE-PCR assay has been verified.

Embryo culture: Late 2-cell stage mouse embryos (B6C3F1 females bred with B6D2F1 males) frozen in straws were obtained from Embryotech Laboratories, Inc. (Wilmington, Mass.) and were handled as described elsewhere³⁰. Briefly, embryos were thawed for 2 minutes at room temperature followed by 1 min at 37° C., and were washed twice in Modified HTF Medium Hepes with 5% Synthetic Serum Substitute (Irvine Scientific, Santa Ana, Calif.) at 37° C. The embryos were kept in the HTF medium for 5 minutes at 37° C. to allow them to rehydrate before being transferred into 35 μl droplets of GEM-PS medium (Duncan Holly Biomedical, Bedford, Mass.) with 5% Synthetic Serum Substitute, overlaid with a very thin layer of Mineral Oil (embryo-tested). The mineral oil was previously washed in Water for Embryo Transfer (Sigma Chemical Company, St, Louis, Mo.) and equilibrated with 7.3% CO₂ at 37° C. After two washes, embryos were transferred into new droplets in groups of 5 or 6, and cultured in the presence of 7.3% CO₂ in order to maintain a pH of 7.3 until the expected stage.

Heat shock: Heat shock was performed on embryos that reached the 8-cell stage after 24 hours of incubation at 37° C. Embryos were transferred to an incubator at 43° C. for 10 minutes, 20 minutes or 30 minutes. Control embryos were not removed from the 37° C. incubator. After heat shock, embryos were allowed to recover at 37° C. for two hours unless described elsewhere. The atmosphere of both incubators contained 7.3% CO₂.

Single cell isolation: Prior to isolating single cells, individual 8-cell stage embryos were placed in a 45 μl Dulbecco's PBS droplet without calcium and magnesium chloride, containing 0.4% polyvinyl pyrrolidone (both from Sigma) covered with a thin layer of mineral oil. After one wash, the embryos were kept in the same buffer for 5 minutes to loosen calcium-mediated connections between cells and facilitate single cell isolation. A hole was ablated in the zona pellucida using a ZILOS-tk™ laser optical system (beam=1480 nm) (Hamilton Thome Biosciences, Inc., Beverly, Mass.) as previously described^{31,32}. Individual cells were extruded sequentially through this hole. The number of cells comprising the embryo was confirmed visually by counting the cells released in the medium.

PurAmp procedure for single cells and whole embryos lysis-to-RT-PCR: PurAmp, a rapid single-tube method developed in our laboratory was used for lysing single cells and whole embryos and for analyzing them by RT-PCR in the same vessel³³. Briefly, LysoDots were prepared in advance by delivering 20-nl aliquots of the denaturing solution to the inside surface of the lids of PCR-grade reaction tubes (Applied Biosystems, Foster City, Calif.) and were stored at room temperature in closed PCR tubes. The denaturing solution composition was as follows: 0.25% sarcosyl, 2 M GITC, 100 mM β-Mercapto-ethanol, 0.01 M sodium citrate, pH 7.0 (all reagents from Stratagene, La Jolla, Calif.), and 1% (v/v) dimethyl sulfoxide (Sigma Chemical Company, St. Louis, Mo.). The single cells or whole embryos were collected as previously described³³ and the lysed samples were heated at 75° C. for 5 minutes, after which they were once again dry or semi-dry. The samples were then stored at −70° C. until used for RT-LATE-PCR.

Measurement of hsp70i RNA copy number by one-step RT-LATE-PCR: All experimental procedures were carried out using rigorous precautions to avoid or destroy environmental RNases. The microscope stage and all the facilities used during the procedure were treated with RNase Out (Sigma,) and wiped with bleach before each experiment. Each dry lysed sample on a PCR tube lid was re-solubilized with 10 μl of water and closed with an inverted PCR tube. The tubes were turned right-side up and their contents were centrifuged to the bottom³³. The samples, containing both DNA and RNA from the starting single cell or embryo were kept on ice until one-step RT-LATE-PCR.

Real-time one-step RT-LATE-PCR was carried out in a final volume of 50 μl, by adding 40 μl of a reaction mixture composed of 60 mM Tris-SO₄ (pH 8.9), 18 mM ammonium sulfate (Invitrogen), 3 mM MgCl₂, 0.4 mM dNTPs, 50 nM limiting primer, 2 μM excess primer, 500 nM probe, 300 nM Primesafe PS002 (Smith Detection, UK)³⁴, and 1 μl of SSIII/Platinum Taq mixture from the SuperScript™ III Platinum® One-Step Quantitative RT-PCR System (Invitrogen, CA). The cycling profile was as follows: 55° C. for 15 minutes (RT); 95° C. for 5 minutes; 15 cycles consisting of the following three steps: 95° C. (10 sec), 63° C. (20 sec) and 72° C. (30 sec); 35 cycles with the following four steps: 95° C. (15 sec), 55° C. (25 sec), 72° C. (35 sec) and 45° C. (30 sec). The limiting primer was 5′-CAGCGTCCTCTTGGCCCTCTCACAC-3′(SEQ ID NO:1), and the excess primer was 5′-GATCGACGACGGCATCTTC-3′(SEQ ID NO:2) for both hsp70i genes. The probe was a molecular beacon with a 2-base pair stem and the following sequence: BHQ1-5′-GATCCTCTTGAACTCCTTC-3′(SEQ ID NO:3)-Cal Orange 560. (BHQ1=Black Hole Quencher 1.) Fluorescence was acquired at 45° C. The probe is a low-Tₘ probe with a Tₘ of 53° C. which does not hybridize to target at 55° C. and above and therefore does not interfere with amplification efficiency.

The primers and probe were designed using VisualOmp (DNAsoftware, Inc., MI) following the LATE-PCR primer/probe design criterion^{35,36}. All oligonucleotides were ordered from Biosearch Technologies (Novato, Calif.). The chosen primers are localized at positions 633/822 of the hsp70.1 GenBank sequence with accession number M35021 and were identical to sequences within the hsp70.3 gene, previously known as hsp70A1 (GenBank sequence with accession umber M76613). Hsp70.1 and hsp70.3 encode almost identical HSP proteins of 70 kDa which protect cells and facilitate their recovery from stress-induced damage³⁷. In each cell, these primers amplify two copies of the hsp70.1 plus two copies of the hsp70.3 genomic DNA targets on the chromosome 17 pair, in addition to the mRNA copies present, because these genes are intronless and, thus, there is no difference between genomic DNA and cDNA templates for PCR³⁸.

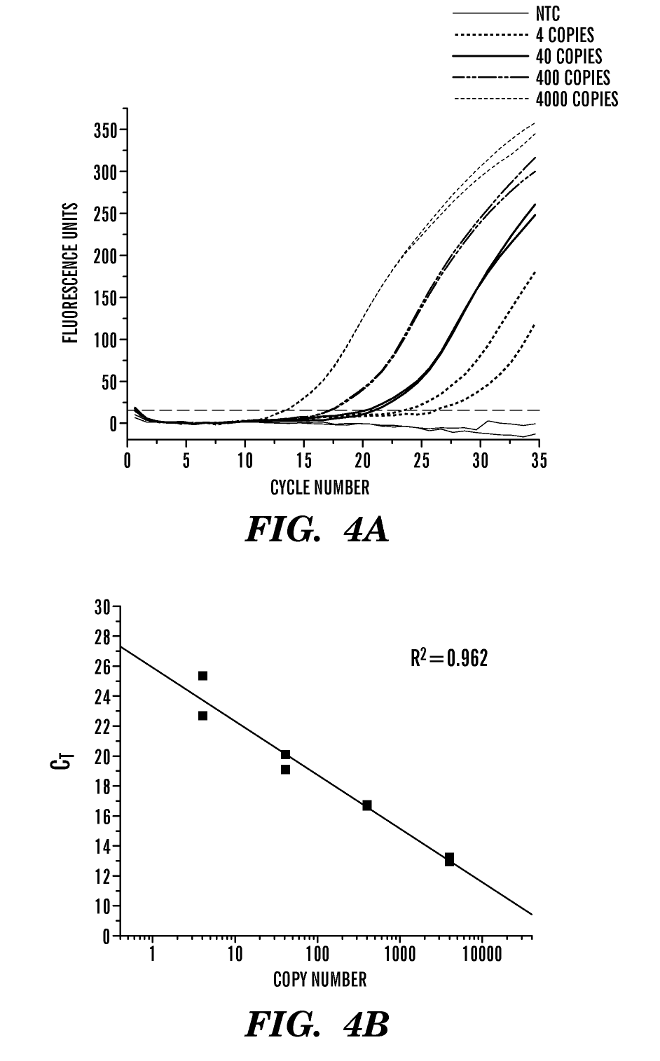

Standard curves for template copy number quantification were generated with the same pair of primers, using a template of purified mouse genomic DNA (Sigma, D4416) in serial dilutions containing the following number of copies: 1,000, 100, 10 and 1. All the LysoDot components were also included in the preparation of the standards. The standard curves were built by correlating the copy numbers of the mouse genome's serial dilutions with the C_{T} values from real-time LATE-PCR (FIG. 4). The C_{T} values obtained from real-time LATE-PCR of experimental samples were then converted into copy number by using these standards. Standards were included in each experiment to account for some batch-to-batch reagents variability.

Verification of the Quantitative Accuracy of One-step RT-LATE-PCR: The average number of hsp70i mRNA+DNA copies per cell was obtained using 30-50 single cells from each group of embryos. Single cells from 8-cell stage embryos were isolated following laser ablation of the zona pellucida and were lysed using the PurAmp single-tube method³³. Our previous study has shown that ablation of the zona pellucida with a laser does not induce heat shock³².

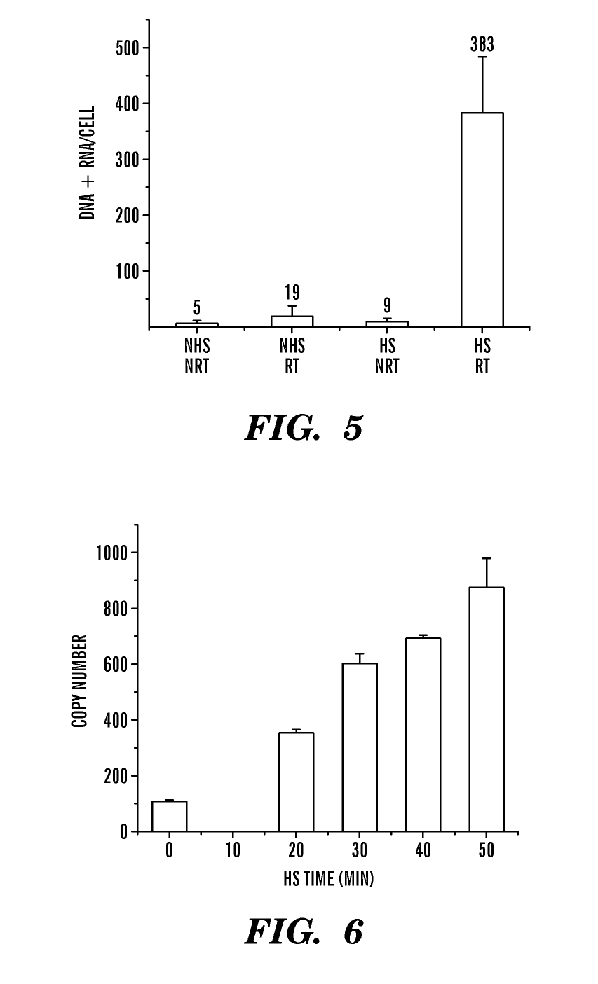

Each diploid genome contains at total of four copies of hsp70.1 plus hsp70.3 and thus each cell contains 4 to 8 gene copies depending on where it is in the cell cycle. FIG. 5 shows that non heat-shocked and heat-shocked single cells contained, respectively, an average of 5 (±5) and 9 (±5) hsp70i template copies when analyzed using our one-step protocol without reverse transcriptase. In this case, all hsp70i templates are genomic DNA and, therefore, this result verifies that the PCR portion of the one-step protocol is sensitive and reliable.

The efficiency of the reverse transcription process was judged by comparing the total number of hsp70i mRNA copies per cell spontaneously expressed or after a 30 minute heat treatment as measured here using the one-step RT-LATE-PCR protocol and as measured previously using a two-step protocol^{32,38,39}. Boonkusol et al have measured the spontaneous hsp70i mRNA transcription in whole 8-cell stage embryos frozen-stored in straws³⁹. Their two-step RT-PCR gives about 63 hsp70i RNA copies per embryo, which is 8 copies per cell, after a 3-hour or 10-hour post-warm. Our previous study obtained 36 copies of hsp70i mRNA per cell from 8-cell stage mouse embryos by two-step RT-PCR. All these number are very close to the number, 19 per cell, obtained from this one-step assay (FIG. 5). After 30 minutes heat shock, using the current one-step protocol, the RNA+DNA copy number was 383 (±100) per cell (FIG. 5). Using the two-step protocol the number was about 500 copies per cell^{32,38}. The slight difference between these two measurements is not significant, given the well established variation of gene expression levels among different embryos^{30,31,33}. The one-step protocol is easier to carry out and may, in fact, be more accurate because two-step protocols that use random hexamers to primer reverse transcription are known to over estimate mRNA levels⁴⁰. This consistency in these numbers obtained by different investigators provides a high level of confidence in their accuracy, as well as the methods used.

### Example 2

### Heat Shock “Memory” Renders Mouse Embryonic Stem Cells More Sensitive to Secondary Stressors

To investigate whether brief heat treatment of mouse embryonic stem (mES) cells can render them more sensitive to secondary heat treatment, we heat-shocked mES cells and measured hsp70i mRNA levels in cells that had been treated one or more times with different doses of heat.

FIG. 6 shows the levels of hsp70i mRNA two hours after heat shock at 43° C. recovered from five equivalent samples containing ˜500 cells (mES) The levels of hsp70i mRNA correlate with the length of heat shock treatment. The longer the heat shock, the higher the hsp70i mRNA levels. In the absence of heat shock there were an average of 105±6 copies hsp70i mRNA. Twenty minutes of heat shock increased the level of hsp70i mRNA 353±14 copies, while 30, 40 and 50 minutes heat shock raised the levels of hsp70i mRNA to 601±34, 691±9 and 875±103 copies, respectively.

To investigate the possibility of a “heat shock memory” in mES cells, we treated four groups of cells as follows. Group 1 was collected without any heat shock. Group 2 was heat shocked for 20 minutes and collected two hours after heat shock when a maximum heat shock response was obtained (Example 1). Group 3 was heat shocked for 20 minutes, then cultured at 37° C. for seven days before the second 20 minutes heat shock was performed, and then collected two hours later. Group 4 was heat shocked for 20 minutes and collected seven days after heat shock without secondary heat shock. Differences in cell number due to cell division over the seven day period were taken into account when analyzing the level of hsp70i mRNA in each sample.

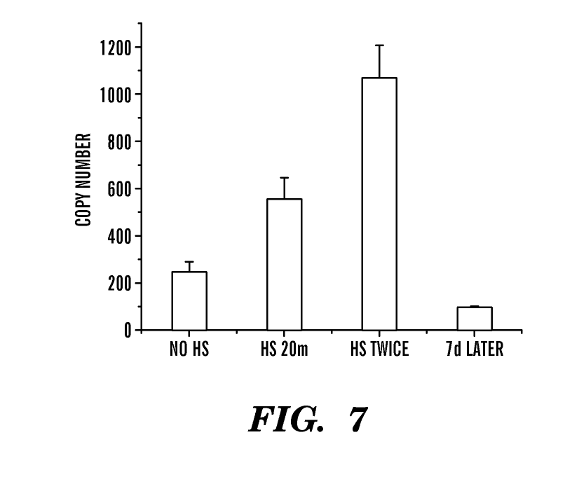

As shown in FIG. 7, an average of 246±41 copies of hsp70i mRNA were measured in the absence of heat shock (Group 1), while a 20-minute heat shock resulted in 555±91 copies (Group 2). Seven days after the first 20 minutes heat shock, the level of hsp70i mRNA was, 94±7 copies (Group 4), which means that there was no mRNA left over from the first heat shock. However, a second 20-minute heat shock seven days after the first heat shock generated 1,070±134 copies (Group 3), almost double the number of hsp70i mRNA molecules produced by a single heat shock. This increased number of mRNA molecules is not left over mRNA due to the first heat shock, or is it dues to a greater number of cells in the sample. Instead, it indicates that some of the cells remembered the experience of the first heat shock and had a higher response to the second heat shock.

In the experiments described above, each sample contained about ˜500 cells. The apparent number of hsp70i mRNA copies per cell is relatively low when calculated on a per cell basis (100-1,000/500 cells=0.2-2 copies/cell). This is likely due to the fact that stem cells in culture are in different phases of the cell cycle. Nevertheless, it can be concluded that responsive cells display a “heat shock memory”.

In conclusion, a “heat shock memory” is observed in mES cells.

### Methods

Cell culture: Mouse embryonic stem cell (mES) line CCE derived from mouse strain 129/Sv was obtained from Stem Cell Technologies (Vancouver, Canada), and adapted to feeder-free conditions (typically mES cells are grown on a mouse embryonic fibroblast layer for secreted nutrients). The cells were grown in Dulbecco's Modified Eagle's Medium (Invitrogen, Carlsbad, Calif.) supplemented with 20% ES-CULT® Fetal Bovine Serum (Stem Cell Technologies, Vancouver, Canada), 100×L-Glutamine, MEM-NEAA, and Pyruvate (Invitrogen, Carlsbad, Calif.) and mouse LIF at a concentration of 1000 units/ml (Millipore, Billerica, Mass.), on 0.1% gelatin-coated 6-well tissue culture plates (Gelatin from Millipore, Billerica, Mass., plates from Nunc-Thermo Scientific, Rochester, N.Y.). The cells were passaged every 2-3 days with 0.25% Trypsin-EDTA (Invitrogen, Carlsbad, Calif.) at split ratios of 1:8-1:12.

Heat shock: Heat shock was performed at 43° C. One confluent well of mES cells (between 1-2 million cells) were placed under media warmed to 43° C. (for efficient and even heat shock treatment), and transferred to an incubator at 43° C. for 20, 30, 40 or 50 minutes. Control mES cells were not removed from the 37° C. incubator. After heat shock treatment, the media was replaced with media at 37° C., and the mES cells were allowed to recover at 37° C. for two hours before collection into Trizol reagent (Invitrogen, Carlsbad, Calif.) or RLT buffer (Qiagen, Valencia, Calif.) for RNA preservation.

RNA extraction: Two methods of RNA extraction were used, Qiagen RNeasy kit for FIG. 6 and Trizol Reagent for FIG. 7. When Trizol Reagent was used, RNA was isolated by adding 1 volume molecular-biology grade chloroform (Sigma, St. Louis, Mo.) per 4 volumes of Trizol, with a 5 minute incubation at room temperature. Samples were spun at 15,000 RCF for 15 minutes in a cooled (4° C.) centrifuge. The top, aqueous layer was collected and precipitated with one volume of molecular-biology grade isopropanol (Sigma, St. Louis, Mo.). The samples were spun down at 15,000 RCF for 10 minutes in a cooled (4° C.) centrifuge. After aspirating the supernatant above the nucleic acid pellet, the pellet was washed with molecular-biology grade ethanol (Sigma, St. Louis, Mo.), and then resuspended in water for further processing. For samples collected in RLT buffer, the Qiagen RNeasy kit was employed according to manufacturer instructions (Qiagen, Valencia, Calif.).

Reverse Transcription: SuperScript III First-Strand Synthesis SuperMix for qRT-PCR (Invitrogen, Carlsbad, Calif.) was used for the reverse transcription. Up to 1 □g of extracted mRNA was added to the RT Enzyme Mix and RT Reaction Mix according to manufacturer's instructions and incubated at 50° C. for 30 minutes to convert mRNA to cDNA, followed by a 5-minute incubation at 85° C. to denature remaining unconverted RNA.

LATE-PCR: Real-time LATE-PCR was carried out in a final volume of 25 □l, by adding 2 □l of diluted cDNA which gave ˜500 cells into 23 □l of a PCR mixture composed of 60 mM Tris-SO₄ (pH8.9), 18 mM ammonium sulfate (Invitrogen), 3 mM MgCl₂, 0.4 mM dNTPs, 50 nM limiting primer, 10M excess primer, 500 nM probe, 200 nM Primesafe PS002 (Smith Detection, UK), and 0.2 □l of Platinum Taq mixture (Invitrogen, CA). The cycling profile was as follows: 95° C. for 5 minutes; 15 cycles consisting of the following three steps: 95° C. (10 sec), 63° C. (20 sec) and 72° C. (30 sec); 35 cycles with the following four steps: 95° C. (15 sec), 55° C. (25 sec), 72° C. (35 sec) and 45° C. (30 sec). The sequence of the limiting primer was 5′-CAGCGTCCTCTTGGCCCTCTCACAC-3′ (SEQ ID NO:1), and that one of the excess primer was 5′-GATCGACGACGGCATCTTC-3′ (SEQ ID NO:2) for both hsp70i genes (Example 1). The probe was a molecular beacon with a 2-base pair stem and the following sequence: BHQ2-5′-GATCCTCTTGAACTCCTTC-3′ (SEQ ID NO:3)-Quasar 670. (BHQ2=Black Hole Quencher 2.) Fluorescence was acquired at 45° C.

Standard curves for template copy number quantification were generated with the same pair of primers, using a template of purified mouse genomic DNA (Sigma, D4416) in serial dilutions containing the following number of copies: 1,000, 100 and 10. Two □l of a “no template” sample that underwent the RNA extraction process and the RT step were included in the preparation of the standards to maintain an identical buffer system for both standards and cDNA samples. The standard curves were built by correlating the copy numbers of the mouse genome's serial dilutions with the CT values from real-time LATE-PCR. The CT values obtained from real-time LATE-PCR of experimental samples were then converted into copy number by using these standards. Standards were included in each experiment to account for some batch-to-batch reagents variability. All the samples were run in duplicates.

### REFERENCES

1. Chandra, A., et al. Fertility, family planning, and reproductive health of U.S. women: data from the 2002 national Survey of Family Growth. Vital Health Stat 23, 1-160 (2005).
2. Ecker, D. J. et al. Long-term effects of culture of preimplantation mouse embryos on behavior. Proc. Natl. Acad. Sci. USA 101, 1595-1600 (2004).
3. Hartshorn, C., et al. Early onset of heat-Shock response in mouse embryos revealed by quantification of stress-inducible hsp70i RNA. Gene Regul Syst Bio 1, 365-373 (2007).
4. matzuk, M. M. & Lamb, D. J. The biology of infertility: research advances and clinical challenges. Nat. Med. 14, 1197-1213 (2008).
5. Nair, P. As IVF becomes more common, some concerns remain. Nat. Med. 14, 1171 (2008).
6. DeBaun, M. R., et al. Association of in vitro fertilization with Beckwith-Wiedemann syndrome and epigenetic alterations of LIT1 and H19. Am J. Hum. Genet. 72, 156-160 (2003).
7. Somjate Manipalviratn, et al. Imprinting disorders and assisted reproductive technology. Fertility and Sterility 91, 305-315 (2009).
8. Cox, G. F. et al. Intracytoplasmic sperm injection may increase the risk of imprinting defects. Am. J. Hum. Genet. 71, 162-164 (2002).
9. Olson, C. K. et al. In vitro fertilization is associated with an increase in major birth defects. Fertil. Steril. 84, 1308-1315 (2005).
10. Reefhuis, J. et al. Assisted reproductive technology and major structural birth defects in the United States. Hum. Reprod. 1, 1-7 (2008).
11. Rinaudo, P. & Schultz, R. M. Effects of embryo culture on global pattern of gene expression in preimplantation mouse embryos. Reproduction 128, 301-311 (2004).
12. Rivera, R. M. et al. Manipulations of mouse embryos prior to implantation result in aberrant expression of imprinted genes on day 9.5 of development. Hum. Mol. Genet. 17, 1-14 (2008).
13. Burel, C. et al. Mammalian heat-shock protein families—expression and functions. Experientia 48, 629-634 (1992).
14. Samali, A. & Cotter, T. G. Heat shock proteins increase resistance to apoptosis. Exp. Cell Res. 223, 163-170 (1996).
15. Pomerai, D. d. et al. Non-thermal heat-shock response to microwaves. Nature 405, 417 (2000).
16. Taylora, R. P. & Benjamin, I. J. Small heat shock proteins: a new classification scheme in mammals J. Mol. Cell. Cardiol. 38, 433-444 (2005).
17. Kapron-Bras, C. M. & Hales, B. F. Heat-shock induced tolerance to the embryotoxic effects of hyperthermia and cadmium in mouse embryos in vitro. Teratology 43, 83-94 (1991).
18. Mirkes, P. E., et al. Heat shock protein 70 (Hsp70) protects postimplantation murine embryos from the embryolethal effects of hyperthermia. Dev. Dyn. 214, 159-170 (1999).
19. Matwee, C., et al. The effects of antibodies to heat shock protein 70 in fertilization and embryo development. Mol. Hum. Reprod. 7, 829-837 (2001).
20. Li, G. C. & Laszlo, A. Amino acid analogs while inducing heat shock proteins sensitize CHO cells to thermal damage. J. Cell. Physiol. 122, 91-97 (1985).
21. Fuller, D. J. M. & Gerner, E. W. Sensitization of chinese hamster ovary cells to heat shock by a-Difluoromethylornithine1. Cancer Res. 47, 816-820 (1987).
22. Wang, C., et al. Heat shock proteins are methylated in avian and mammalian cells. Proc. Natl. Acad. Sci. U.S.A. 78, 3531-3535 (1981).
23. Hartshorn, C., et al. Developmentally-regulated changes of Xist RNA levels in single preimplantation mouse embryos, as revealed by quantatitive real-time PCR. Mol. Reprod. Dev. 61, 425-436 (2002).
24. Hartshorn, C., et al. Laser zona drilling does not induce hsp70i transcription in blastomeres of eight-cell mouse embryos. Fertil. Steril. 84, 15471550 (2005).
25. Hartshorn, C., et al., L. J. Rapid, single-tube method for quantitative preparation and analysis of RNA and DNA in samples as small as one cell. BMC Biotechnol. 5, 2 (2005).
26. Sanchez, J. A., et al. Linear-After-The-Exponential (LATE)-PCR: An advanced method of asymmetric PCR and its uses in quantitative real-time analysis. Proc. Natl. Acad. Sci. USA 101, 1933-1938 (2003).
27. Pierce, K. E., et al. Linear-After-The-Exponential (LATE)-PCR: Primer design criteria for high yields of specific singlestranded DNA and improved real-time detection. Proc. Natl. Acad. Sci. USA 102, 8609-8614 (2005).
28. Rice, J. E. et al. Monoplex/multiplex linear-after-the-exponential-PCR assays combined with PrimeSafe and Dilute-‘N’-Go sequencing. Nat Protoc 2, 2429-2438 (2007).
29. Kiessling, A., et al. Evidence that human blastomere cleavage is under unique cell cycle control. J. Assist Reprod Genet DOI 10.1007/s10815-009-9306
30. Hartshorn, C., et al. Developmentally-regulated changes of Xist RNA levels in single preimplantation mouse embryos, as revealed by quantatitive real-time PCR. Mol. Reprod. Dev. 61, 425-436 (2002).
31. Hartshorn, C., et al. Differential pattern of Xist RNA accumulation in single balstomeres isolated from 8-cell stage mouse embryos following laser zona drilling. Mol. Reprod. Dev. 64, 41-51 (2003).
32. Hartshorn, C., et al. Laser zona drilling does not induce hsp70i transcription in blastomeres of eight-cell mouse embryos. Fertil. Steril. 84, 15471550 (2005).
33. Hartshorn, C., et al. Rapid, single-tube method for quantitative preparation and analysis of RNA and DNA in samples as small as one cell. BMC Biotechnol. 5, 2 (2005).
34. Rice, J. E. et al. Monoplex/multiplex linear-after-the-exponential-PCR assays combined with PrimeSafe and Dilute-‘N’-Go sequencing. Nat Protoc 2, 2429-2438 (2007).
35. Sanchez, J. A., et al. Linear-After-The-Exponential (LATE)-PCR: An advanced method of asymmetric PCR and its uses in quantitative real-time analysis. Proc. Natl. Acad. Sci. USA 101, 1933-1938 (2003).
36. Pierce, K. E., et al. Linear-After-The-Exponential (LATE)-PCR: Primer design criteria for high yields of specific singlestranded DNA and improved real-time detection. Proc. Natl. Acad. Sci. USA 102, 8609-8614 (2005).
37. Huang, L., et al. Insights into regulation and function of the major stress-Induced hsp70 molecular chaperone in vivo: analysis of mice with targeted gene disruption of the hsp70.1 or hsp70.3 Gene. Mol. Cell. Biol. 21, 8575-8591 (2001).
38. Hartshorn, C., et al. Early onset of heat-Shock response in mouse embryos revealed by quantification of stress-inducible hsp70i RNA. Gene Regul Syst Bio 1, 365-373 (2007).
39. Boonkusol, D. et al. Gene expression profiles and in vitro development following vitrification of pronuclear and 8-cell stage mouse embryos. Mol. Reprod. Dev. 73, 700-708 (2006).
40. Bustin, S. A., et al. Quantitative real-time RT-PCR—a perspective. J. Mol. Endocrinol. 34, 597-601 (2005).

All references described herein and throughout the specification are incorporated by reference in their entirety.

1. A method for making a sensitized embryo or embryonic cell comprising treating a cleavage staged embryo or embryonic cell arising from said embryo with a stress inducer such that a level of at least one heat shock protein or mRNA is increased to a sufficient level to exhibit a greater response to a second exposure to the stress inducer.

2. The method of claim 1, wherein the heat shock protein is selected from the group consisting of: Hsp10, Hsp27, HspB1, Hsp40, Hsp60, Hsp71, Hsp70, Hsp72, Grp78 (BiP), Hsx70, HSP70i, Hsp90, Grp94, Hsp104, and Hsp110.

3. The method of claim 1, wherein the stress inducer is applied in an in vitro culture or applied in vivo.

4. (canceled)

5. The method of claim 1, wherein the embryo or embryonic cell is not a human embryo or a human embryonic cell.

6. The method of claim 1, wherein said stress inducer is a heat shock.

7. (canceled)

8. The method of claim 1, wherein the stress inducer is applied for 10-25 minutes.

9. (canceled)

10. The method of claim 1, further comprising a step of measuring the level of a heat shock protein after treating with a stress-inducer, wherein the level of heat shock protein is measured by a method selected from the group consisting of: assessing the level of heat shock protein mRNA, assessing the level of heat shock protein, and assessing the level of a visibly detectable protein operably linked to a heat shock protein promoter.

11. (canceled)

12. (canceled)

13. The method of claim 1, wherein the stress inducer is a chemical stress inducer.

14. The method of claim 13, wherein said chemical stress inducer is selected from the group consisting of: bimoclomol (BIM), 2-Cyclopenten-1-one, thapsigargin; tunicamycin, a geldanamycin derivative, zinc, an amino acid analog, α-difluoromethylornithine, a household chemical, and an environmental pollutant.

15. The method of claim 1, further comprising subjecting said cleavage staged embryo or embryonic cell arising from said embryo to a second treatment with a stress inducer.

16. The method of claim 15, wherein the second treatment is performed using the same stress inducer or using a different stress inducer.

17. (canceled)

18. The method of claim 1 wherein said second treatment is given in vivo or in vitro.

19. A method for making a sensitized test mammal comprising
a. treating a cleavage staged embryo of a mammal with a stress inducer such that the levels of a heat shock protein or mRNA are increased to a sufficient level to exhibit a greater response to a second treatment with a stress inducer, wherein when the stress inducer is heat shock, the heat shock is applied in vitro;
b. developing the cleavage staged embryo of step a) for implantation; and
c. implanting the embryo of step b) into the uterus of a mammal for in-vivo growth and birth, whereby the birthed mammal is the sensitized test mammal that exhibits a sensitized response to a secondary stress inducer.

20. The method of claim 19, further comprising subjecting said cleavage staged embryo to a second treatment with a stress inducer, wherein said second treatment is performed in vitro or in vivo.

21. The method of claim 19, wherein the second treatment with a stress inducer is performed using the same stress inducer or using a different stress inducer.

22. (canceled)

23. (canceled)

24. A method for making a sensitized test non-mammalian animal comprising
a. treating a cleavage staged embryo with a stress inducer such that the levels of a heat shock protein or mRNA are increased to a sufficient level to exhibit a greater response to a second treatment with a stress inducer;
b. developing the cleavage staged embryo of step a) to an advanced stage of development and multicellular differentiation,
whereby the multicellular animal exhibits a sensitized response to a secondary stress inducer.

25. The method of claim 24, further comprising subjecting said cleavage staged embryo to a second treatment with a stress inducer, wherein said second treatment is performed in vitro at a later stage of development.

26. The method of claim 24, wherein the second treatment with a stress inducer is performed using the same stress inducer or using a different stress inducer.

27. (canceled)

28. A method for screening for compounds that inhibit induction of stress by a stress inducer comprising
a. contacting a test model selected from a sensitized test animal of claim 19 or a sensitized embryo or embryonic cell with a test compound,
b. contacting the test model of step a) with a stress inducer,
c. assessing the level of heat shock protein or mRNA in cells of the test model of step b); and
d. comparing the level of heat shock protein or mRNA determined in step c) to a control level in cells of the corresponding test model not treated with the test compound, wherein a decreased level of heat shock protein or mRNA when compared to the control level indicates that the test compound is compounds that inhibits induction of stress by the stress inducer.

29. A method for screening for compounds that induce stress in a cell comprising
a. contacting a test model selected from the sensitized test mammal or non-mammal of claim 19 and a sensitized embryo and embryonic cell with a test compound,
b. contacting the test model of step a) with a stress inducer,
c. detecting the level of heat shock protein or mRNA in cells of the test model step b); and
d. comparing the level of heat shock protein or mRNA determined in step c) to a control heat shock protein or mRNA level in cells of the corresponding test model not treated with the test compound,
wherein an increased level of heat shock protein or mRNA as compared to the control level indicates that the test compound is a compound that induce stress in the cell.
